(19) United States
(12) Patent Application Publication
(10) Pub. No.: US 2022/0409234 A1
(43) Pub. Date: Dec. 29, 2022

(54) SERIAL DILATION SYSTEM

(71) Applicant: Medos International Sarl, Le Locle (CH)

(72) Inventors: Alicia McDERMOTT, Waltham, MA (US); Paul S. MAGUIRE, Hope Valley, RI (US); Andrew E. MEDEIROS, Westport, MA (US); Jessica INDYK, Canton, MA (US); Phat LUU, New Bedford, MA (US)

(21) Appl. No.: 17/355,774

(22) Filed: Jun. 23, 2021

(51) Int. Cl. A61B 17/34; A61M 29/00

(52) U.S. Cl. CPC A61B 17/3423; A61M 29/00; A61B 2017/3433

(57) ABSTRACT

A serial dilation system includes a plurality of serial dilators that can be advanced over each other so as to dilate anatomical soft tissue along a trajectory toward a target anatomical site. An access cannula can then be introduced over the serial dilators. The serial dilators can be coupled to each other, such that once the access cannula is in place, removal of one of the serial dilators from the access cannula causes all of the serial dilators to be removed from the access cannula.

### BACKGROUND

The human spine is comprised of a series of vertebral bodies separated by intervertebral discs. The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus within its cushioning qualities. These nucleus pulposus cells may also secrete small amounts of cytokines such as interleukin-1.beta. and TNF-.alpha. as well as matrix metalloproteinases (“MMPs”). These cytokines and MMPs help regulate the metabolism of the nucleus pulposus cells.

In some instances of degenerative disc disease (DDD), gradual degeneration of the intervertebral disc is caused by mechanical instabilities in other portions of the spine. In these instances, increased loads and pressures on the nucleus pulposus cause the cells within the disc (or invading macrophages) to emit larger than normal amounts of the above-mentioned cytokines. In other instances of DDD, genetic factors or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of proinflammatory cytokines and/or MMPs that may cause nerve irritation and pain.

As DDD progresses, toxic levels of the cytokines and MMPs present in the nucleus pulposus begin to degrade the extracellular matrix. In particular, the MMPs (as mediated by the cytokines) begin cleaving the water-retaining portions of the proteoglycans, thereby reducing their water-retaining capabilities. This degradation leads to a less flexible nucleus pulposus, and so changes the loading pattern within the disc, thereby possibly causing delamination of the annulus fibrosis. These changes cause more mechanical instability, thereby causing the cells to emit even more cytokines, typically thereby upregulating MMPs. As this destructive cascade continues and DDD further progresses, the disc begins to bulge (“a herniated disc”), and then ultimately ruptures, causing the nucleus pulposus to contact the spinal cord and produce pain.

One proposed method of managing these problems is to remove the problematic disc from the intervertebral space and replace it with a porous device that restores disc height and allows for bone growth therethrough for the fusion of the adjacent vertebrae. These devices are commonly called “fusion devices”.

Conventional systems for accessing the intervertebral space can include dilators having progressively increasing sizes that are inserted one over the other to widen an access path that extends along a desired trajectory toward the intervertebral space. A working access cannula is then placed over the largest outermost dilator, and the dilators are then removed while maintaining the access cannula in place. Instruments can be driven through the access cannula to perform the surgical procedure.

### SUMMARY

In one example, a serial dilation system for orthopedic surgery can include a first dilator member and a second dilator member. The first dilator member can be configured to be driven into anatomical soft tissue, such that a first external surface of the first dilator member dilates an opening in anatomical soft the tissue. The second dilator member can be configured to be advanced along the first external surface in a distal direction and through the anatomical soft tissue, such that a second external surface of the second dilator member further dilates the anatomical soft tissue. Movement of one of the first and second dilator members in a proximal direction opposite the distal direction causes respective first and second stop surfaces of the first and second dilator members to abut each other, thereby causing the other of the first and second dilator members to move in the proximal direction along with the one of the first and second dilator members.

### BRIEF DESCRIPTION OF THE DRAWINGS

The foregoing summary, as well as the following detailed description of illustrative embodiments, will be better understood when read in conjunction with the appended drawings. For the purposes of illustrating the present disclosure, there is shown in the drawings illustrative embodiments. It should be understood, however, that the application is not limited to the precise arrangements and instrumentalities shown. In the drawings:

FIG. 1 is a lateral elevational view of a portion of a vertebral column.

FIG. 2 is a schematic side view of Kambin's triangle.

FIG. 3A is a perspective view of a guide member driven into an intervertebral space through Kambin's triangle along a predetermined desired trajectory;

FIG. 3B is a sectional side elevation view of an access assembly including the guide member of FIG. 3A and a serial dilation system that includes a plurality of dilator members and an access cannula;

FIG. 3C is a sectional side elevation view of the access assembly of FIG. 3B, showing the access cannula driven over an outer dilator member of the plurality of dilator members;

FIG. 3D is a sectional side elevation view of the access cannula after removal of the serial dilators and the guide member;

FIG. 4 is a perspective view of the serial dilation system illustrated in FIG. 3B;

FIG. 5A is a side elevation view of an inner dilator member of the serial dilation system illustrated in FIG. 4;

FIG. 5B is a sectional side elevation view of the inner dilator member illustrated in FIG. 5A;

FIG. 5C is an enlarged sectional side elevation view of a portion of the inner dilator member illustrated in FIG. 5B;

FIG. 6A is a side elevation view of a middle dilator member of the serial dilation system illustrated in FIG. 4;

FIG. 6B is a sectional side elevation view of the middle dilator member illustrated in FIG. 6A;

FIG. 6C is an enlarged sectional side elevation view of a portion of the middle dilator member of FIG. 6B;

FIG. 6D is an enlarged sectional side elevation view showing the inner dilator member received by the middle dilator member;

FIG. 7A is a side elevation view of an outer dilator member of the serial dilation system illustrated in FIG. 4;

FIG. 7B is a sectional side elevation view of the outer dilator member illustrated in FIG. 7A;

FIG. 7C is an enlarged sectional side elevation view of a portion of the outer dilator member of FIG. 7B;

FIG. 7D is an enlarged sectional side elevation view showing the inner dilator member received by the middle dilator member, and the middle dilator received by the outer dilator member;

FIG. 8A is a sectional side elevation view of a serial dilation system constructed in accordance with another example;

FIG. 8B is an enlarged sectional side elevation view of a portion of the serial dilation system of FIG. 8A;

FIG. 9A is an exploded perspective view of a serial dilation system constructed in accordance with yet another example;

FIG. 9B is a cross-sectional view of the serial dilation system of FIG. 9A;

FIG. 9C is a sectional side elevation view of the serial dilation system of FIG. 9A, showing an inner dilator member, a middle dilator member, and an outer dilator member spaced from each other;

FIG. 9D is a sectional side elevation view of the serial dilation system of FIG. 9C, showing the inner dilator member, a middle dilator member, and outer dilator member coupled to each other for removal in a single step;

FIG. 10A is a perspective view of a serial dilation system constructed in accordance with still another example;

FIG. 10B is a sectional side elevation view of the serial dilation system of FIG. 10A, showing an inner dilator member, a middle dilator member, and an outer dilator member spaced from each other;

FIG. 10C is a sectional side elevation view of the serial dilation system of FIG. 10B, showing the inner dilator member, a middle dilator member, and outer dilator member coupled to each other for removal in a single step;

FIG. 10D is a sectional side elevation view of the serial dilation system similar to FIG. 10C, but constructed in accordance with another embodiment;

FIG. 11A is an exploded perspective view of a serial dilation system constructed in accordance with still another example; and

FIG. 11B is a sectional side elevation view of the serial dilation system of FIG. 11A.

### DETAILED DESCRIPTION OF ILLUSTRATIVE EMBODIMENTS

Certain terminology is used in the following description for convenience only and is not limiting. The “lower” and “upper” designate directions in the drawings to which reference is made. The words, “anterior”, “posterior”, “superior,” “inferior,” “medial,” “lateral,” and related words and/or phrases are used to designate various positions and orientations in the human body, but also applies to the fusion cage when disposed outside the human body. The terminology includes the above-listed words, derivatives thereof and words of similar import.

Unless otherwise indicated, the terms “substantially,” “generally,” and “approximately” along with derivatives thereof and words of similar import as used herein with respect to dimensions, values, shapes, directions, and other parameters can include the stated dimensions, values, shapes, directions, and other parameters and up to plus or minus 10% of the stated dimensions, values, shapes, directions, and other parameters, such as up to plus or minus 9% of the stated dimensions, values, shapes, directions, and other parameters, such as up to plus or minus 8% of the stated dimensions, values, shapes, directions, and other parameters, such as up to plus or minus 7% of the stated dimensions, values, shapes, directions, and other parameters, such as up to plus or minus 6% of the stated dimensions, values, shapes, directions, and other parameters, such as up to plus or minus 5% of the stated dimensions, values, shapes, directions, and other parameters, such as up to plus or minus 4% of the stated dimensions, values, shapes, directions, and other parameters, such as up to plus or minus 3% of the stated dimensions, values, shapes, directions, and other parameters, such as up to plus or minus 2% of the stated dimensions, values, shapes, directions, and other parameters, such as up to plus or minus 1% of the stated dimensions, values, shapes, directions, and other parameters.

Method steps and apparatus described or referenced herein will sometimes be described in singular form for clarity. However, it should be appreciated that as used herein, the singular term “a” or “the” with respect to an apparatus or method step can include the plural apparatus or method steps. Conversely, the plural term as used herein with respect to apparatus or method steps can include the singular “a” or “the.” Thus, it should be appreciated that the use herein of the singular term “a” or “the” and the use herein of the plural term can equally apply to “at least one” unless otherwise indicated.

In accordance with certain embodiments disclosed herein, an improved serial dilation system is provided for accessing an intervertebral space. For example, in one embodiment, the system includes a plurality of dilation members of increasing size that can be placed over each other to minimally invasively dilate tissue for the subsequent insertion of an access cannula that defines a working channel toward the intervertebral space. Once the access cannula is in place, the dilation members can be removed in fewer steps than conventionally achieved, thereby reducing the number of steps associated with the surgical procedure. Surgical instruments and/or one or more intervertebral implants can then be inserted through the access cannula in a minimally invasive procedure to reduce trauma to the patient and thereby enhance recovery and improve overall results. By minimally invasive, Applicant means a procedure performed percutaneously through an access device in contrast to a typically more invasive open surgical procedure.

Certain embodiments disclosed herein are discussed in the context of an intervertebral implant and spinal fusion because of the device and methods have applicability and usefulness in such a field. The device can be used for fusion, for example, by inserting an intervertebral implant to properly space adjacent vertebrae in situations where a disc has ruptured or otherwise been damaged. “Adjacent” vertebrae can include those vertebrae originally separated only by a disc or those that are separated by intermediate vertebra and discs. Such embodiments can therefore be used to create proper disc height and spinal curvature as required in order to restore normal anatomical locations and distances. However, it is contemplated that the teachings and embodiments disclosed herein can be beneficially implemented in a variety of other operational settings, for spinal surgery and otherwise.

As context for the methods and devices described herein, FIG. 1 is a lateral view of a vertebral column 10. As shown in FIG. 1, the vertebral column 10 comprises a series of alternative vertebrae 11 and fibrous intervertebral discs 12 that provide axial support and movement to the upper portions of the body. The vertebral column 10 typically comprises thirty-three vertebrae 11, with seven cervical (C1-C7), twelve thoracic (T1-T12), five lumbar (LI-LS), five fused sacral (S1-S5), and four fused coccygeal vertebrae. Adjacent vertebrae 11 define respective intervertebral spaces 13 that contain fibrous intervertebral discs 12. At least a portion or an entirety of the fibrous intervertebral disc 12 can be removed from an intervertebral space 13 in preparation for the insertion of an intervertebral implant.

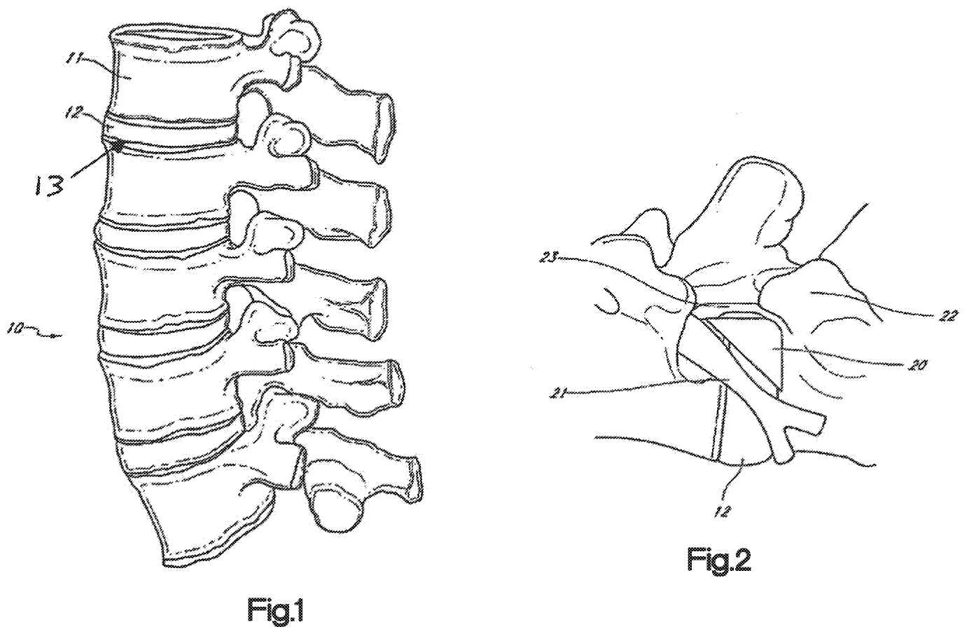

FIG. 2 is a schematic view of Kambin's triangle. This region 20 is the site of posterolateral access for spinal surgery. It can be defined as a right triangle over the intervertebral disc 12 viewed dorsolaterally. The hypotenuse is the exiting nerve 21, the base is the superior border of the inferior vertebra 22, and the height is the traversing nerve root 23. As will be explained below, in one embodiment, the intervertebral disc 12 is accessed through this region by performing a foraminoplasty in which a portion of the inferior vertebra is removed such that surgical instruments or implants can be introduced at this region of the spine. In such a procedure, it is often desired to protect the exiting nerve and the traversing nerve root. Apparatuses and methods for accessing the intervertebral disc through Kambin's triangle may involve performing endoscopic foraminoplasty while protecting the nerve will be discussed in more detail below. Utilizing foraminoplasty to access the intervertebral disc through Kambin's triangle can have several advantages (e.g., less or reduced trauma to the patient) as compared to accessing the intervertebral disc posteriorly or anteriorly as is typically done in the art. In particular, surgical procedures involving posterior access often require removal of the facet joint. For example, transforaminal interbody lumbar fusion (TLIF) typically involves removal of one facet joint to create an expanded access path to the intervertebral disc. Removal of the facet joint can be very painful for the patient, and is associated with increased recovery time. In contrast, accessing the intervertebral disc through Kambin's triangle may advantageously avoid the need to remove the facet joint. As described in more detail below, endoscopic foraminoplasty may provide for expanded access to the intervertebral disc without removal of a facet joint. Sparing the facet joint may reduce patient pain and blood loss associated with the surgical procedure. In addition, sparing the facet joint can advantageously permit the use of certain posterior fixation devices which utilize the facet joint for support (e.g., trans-facet screws, trans-pedicle screws, and/or pedicle screws). In this manner, such posterior fixation devices can be used in combination with interbody devices inserted through the Kambin's triangle.

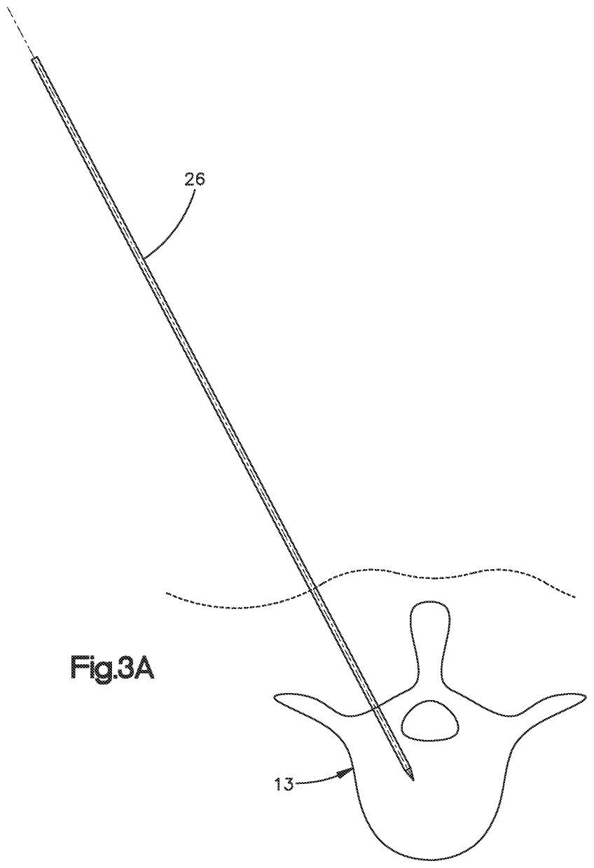

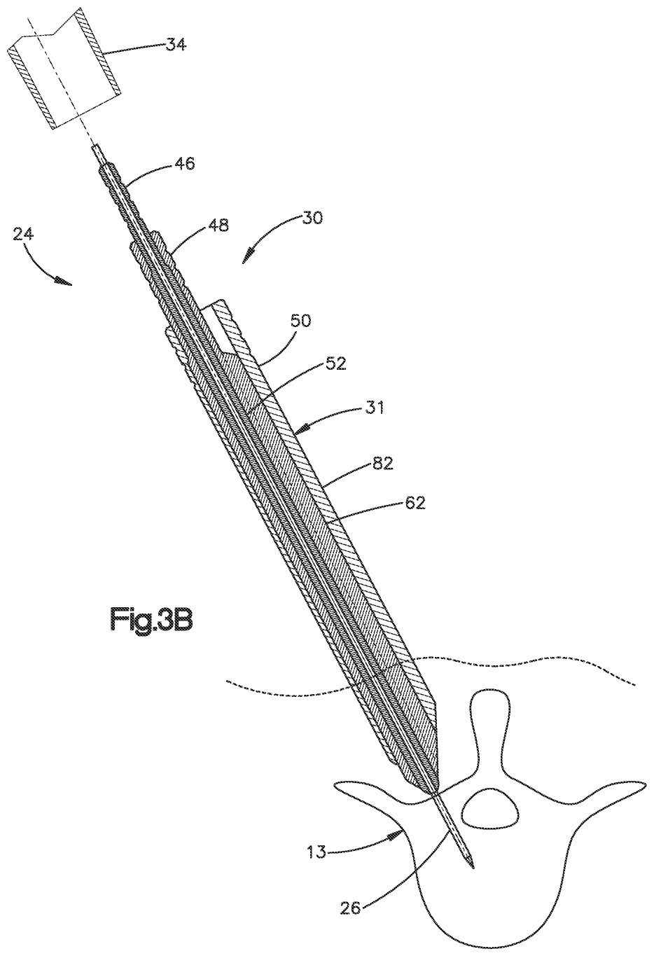

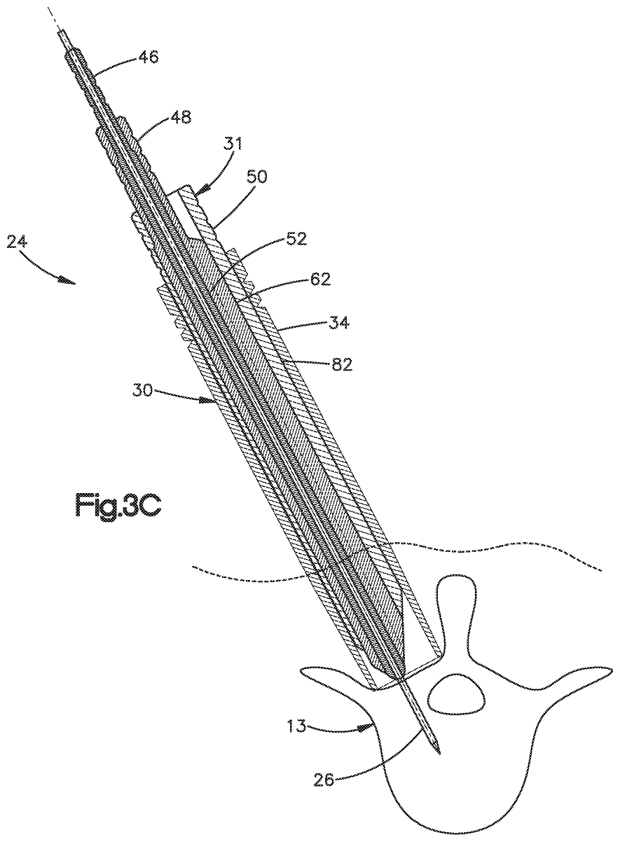

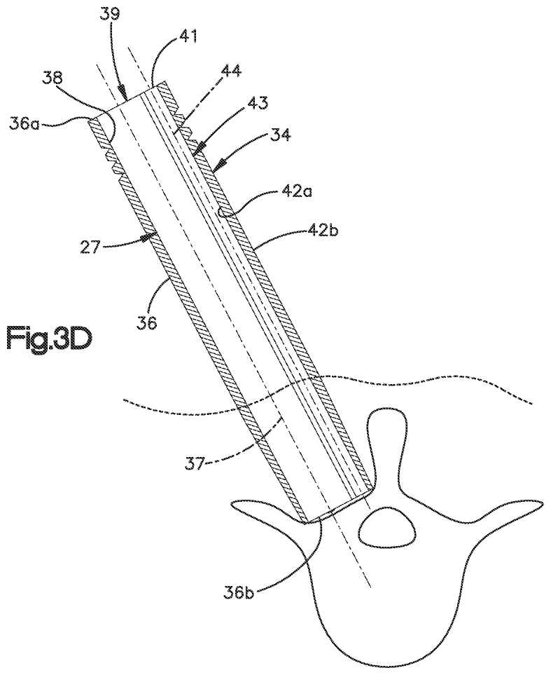

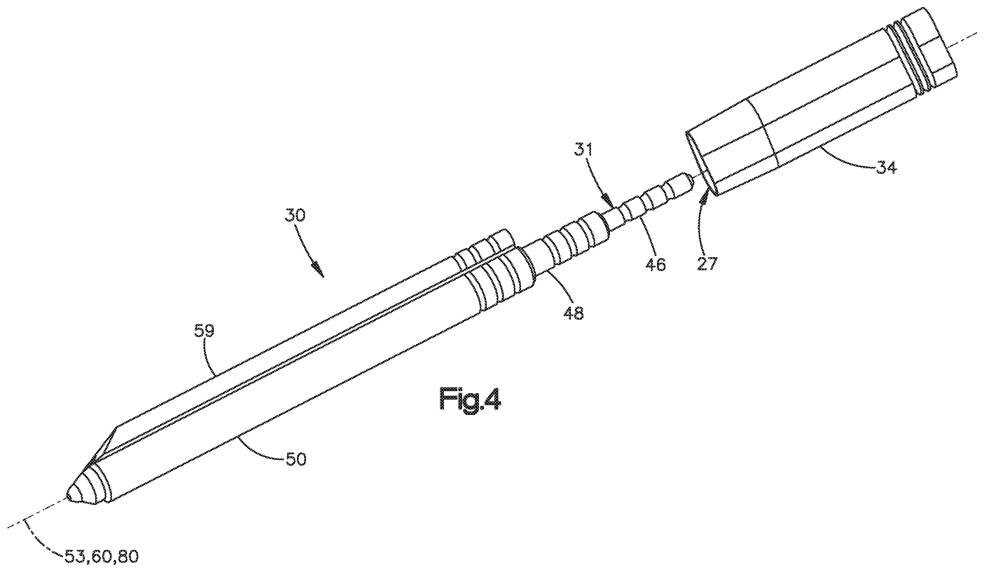

Referring to FIGS. 3A-4, an access assembly 24 can include a guide member 26 and a serial dilation system 30. The serial dilation system 30 can include at least one serial dilator 31 such as a plurality of serial dilators 31 and an access cannula 34. The serial dilation system 30 constructed in one example can be used to perform percutaneous orthopedic surgery. The serial dilators 31, and thus the serial dilation system 30, can include at least an inner dilator member 46, a middle dilator member 48, and an outer dilator member 50. The guide member 26 can be introduced along a desired predetermined trajectory through anatomical soft tissue toward a target anatomical site. While the target anatomical site can be the intervertebral space 13 through the Kambin's triangle as described above, the present invention is not limited to spinal applications, but rather can include any alternative surgical procedure that benefits from serial dilation toward the target anatomical site. For instance, in some examples, the target anatomical site can be a pedicle. Thus, the guide member 26 can extend to the pedicle, and in some examples can threadedly purchase with the pedicle. Accordingly, the serial dilation system 30 can be directed toward a pedicle to provide access for the insertion and fixation of a pedicle screw to the pedicle through a working channel that is defined by the access cannula 34. The desired trajectory can be determined under any suitable guidance as desired. The guide member 26 can be configured as a Jamshidi needle, trocar, Kirshner wire (or K-wire), or the like.

The serial dilation system 30 can be preassembled such that the inner dilator member 46 is received by the middle dilator member 48, which in turn is received by the outer dilator member 50. The outer dilator member 50 defines an external dilation surface 82 that can be sized larger than that an external dilation surface 62 of the middle dilator member 48, which can be sized larger than an external dilation surface 52 of the inner dilator member 46. The inner dilator member 46 is driven distally over the guide member 26. Thus, the guide member 26 guides the inner dilator member 46 to travel in a distal direction along the desired trajectory toward the target anatomical site. As the inner dilator member 46 travels in the distal direction, the inner dilator member 46 enlarges an opening in the anatomical soft tissue that was created by the guide member 26. In other examples, the inner dilator member 46 can define the guide member 26, and thus can create and enlarge the opening in the soft tissue. When the inner dilator member 46 travels over the guide member 26, the inner dilator member 46 enlarges or dilates a previously-created opening. When the inner dilator member 46 creates the opening in the anatomical soft tissue, it can be said that the inner dilator member 46 enlarges or dilates an opening in the anatomical soft tissue that is created by the inner dilator member 46.

The middle dilator member 48 can be driven through the tissue over the inner dilator member 46 in the distal direction to further enlarge the opening. Because the external dilation surface 62 of the middle dilator member 48 is sized larger than the external dilation surface 52 of the inner dilator member 46, the middle dilator member 48 further dilates the anatomical soft tissue as it is driven into the anatomical soft tissue. The outer dilator member 50 can be driven through the tissue over the middle dilator member 48 in the distal direction to still further enlarge the opening. Because the external dilation surface 82 of the outer dilator member 50 is sized larger than the external dilation surface 62 of the middle dilator member 48, the outer dilator member 50 further dilates the anatomical soft tissue as it is driven into the anatomical soft tissue. Finally, the access cannula 34 can be driven in the distal direction over the outer dilator member 50. The guide member 26 and the serial dilation system 30 can then be removed, leaving the access cannula 34 in place. The access cannula 34 thus defines a working channel 27 to the intervertebral space 13. In other examples, as described above, the access cannula 34 of all of the serial dilation systems described herein can define a working channel to a pedicle as desired.

As illustrated in FIG. 3D, the access cannula 34 is shown in position for performing surgery on an intervertebral disc, such as a transforaminal lumbar interbody fusion. The access cannula 34 in the illustrated embodiment can extend through Kambin's triangle in some examples. The access cannula 34 has a tubular body portion 36 that extends along a respective first central cannula axis 37, and a radial bump-out 41 disposed adjacent the tubular body portion 36 along a radial direction that is oriented perpendicular with respect to the first central cannula axis 37. The tubular body portion 36 includes an internal surface 38 that defines an internal lumen 39, and an external surface 40 that is opposite the internal surface 38. The internal lumen 39 can extend through an entirety of the tubular body portion 36 from a proximal end 36a to a distal end 36b of the tubular body portion 36, and can define the working channel 27 of the access cannula 34. The access cannula 34 can be driven over the outer dilator member 50 toward the intervertebral space, such that the internal lumen 39 receives the outer dilator member 50.

Once the access cannula 34 is in place, the dilator members 46-50 can be removed from the internal lumen 39 of the access cannula 34 in a proximal direction opposite the distal direction. Advantageously, two or more up to all of the dilator member, such as the inner dilator member 46 and the outer dilator member 50, can be coupled with respect to proximal movement, such that removal of one of the dilator members 46-50 from the access cannula 34 in the proximal direction can cause one or more others of the dilator members 46-50 to be removed from the access cannula 34 along with the one of the dilator members 46-50. Thus, more than one up to all of the dilator members 46-50 can be removed in a single step. While the illustrated embodiment includes three dilators, other embodiments can include a lesser number or greater number of dilators as desired. Thus, in one example the dilation system 30 can include only the inner dilator member 46 and the outer dilator member 50, which is configured to directly couple with the inner dilator member with respect to movement in the proximal direction. In other examples, the dilation system 30 can include one or more middle dilator members in combination with the inner dilator member 46 and the outer dilator member 50.

Once the serial dilators 31 have been removed from the access cannula 34, the internal lumen 39 can define the working channel 27 that allows for surgical instruments and devices to pass through it to access the intervertebral space 13. The instruments can be configured to remove at least a portion or an entirety of the fibrous disc 12 from the intervertebral space 13, and prepare the intervertebral space 13 to receive an intervertebral implant which is also implanted through the internal lumen 39. The internal lumen can also receive an instrument that is configured to vertically expand the intervertebral implant. Thus, the implant can be inserted through the internal lumen in a contracted position, and subsequently expanded inside the intervertebral space 13. The distal tip of the cannula can be oriented such that surgical instruments have access to the intervertebral space 13 without contacting the exiting nerve. The position shown in FIG. 3D can be achieved by following the method disclosed herein, discussed in more detail below.

The radial bump-out 41 of the access cannula 34 includes an internal bump-out surface 42a that defines an internal bump-out lumen 43, and an external adjacent bump-out surface 42b that is opposite the internal surface 42a. The internal bump-out lumen 43 can extend through an entirety of the radial bump-out 41 from a respective proximal end 41a to a respective distal end 41b of the radial bump-out 41 along a second central cannula axis 44 that can be oriented substantially parallel to the first central cannula axis 37. The first and second axes 37 and 44 can be oriented along a longitudinal direction. The internal bump-out lumen 43 can be configured to receive a camera for viewing the surgical steps performed on and in the intervertebral disc space. The internal bump-out lumen 43 can be open or closed to the internal lumen 39 of the tubular body portion 36 along the radial direction. The tubular body portion 36 and the radial bump-out 41 can impart an asymmetrical shape to the access cannula 34 in a cross-sectional plane that is oriented perpendicular to either or both of the first and second central cannula axes 37 and 44.

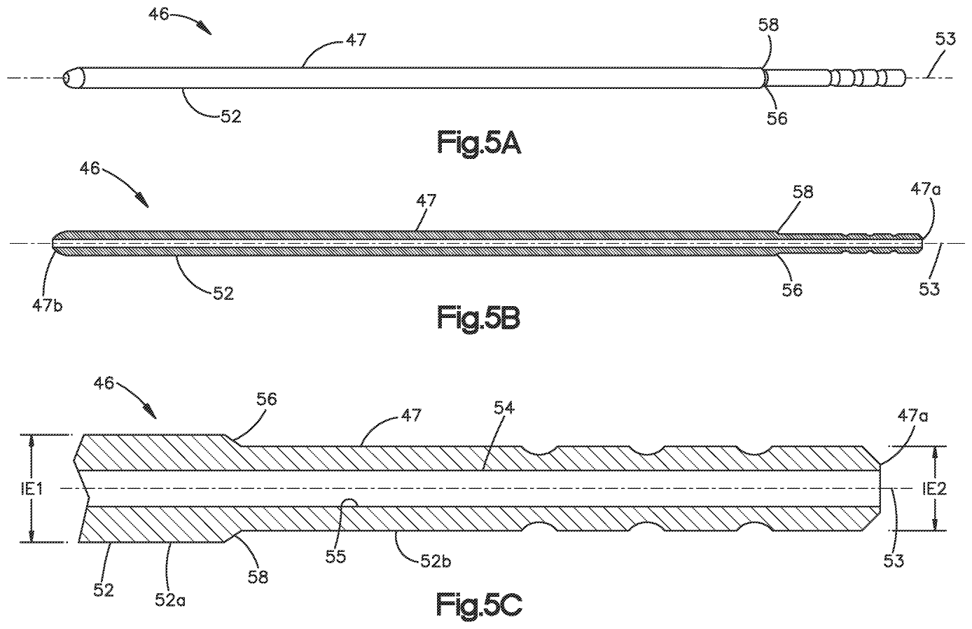

Each of the dilator members 46, 48, and 50 will now be described in detail with reference to FIGS. 5A-7D. For instance, referring now to FIGS. 5A-5C, the inner dilator member 46 includes an inner dilator body portion 47, which can be configured as an inner dilator tube, that extends along an inner central axis 53. The inner central axis 53 extends from a proximal end 47a of the inner dilator body portion 47 to a distal end 47b of the inner dilator body portion 47. Thus, a proximal direction is established from the distal end 47b to the proximal end 47a. A distal direction is established from the proximal end 47a to the distal end 47b. A longitudinal direction can include both the proximal direction and the distal direction. The distal end 47b can be referred to as a leading end of the inner dilator member 46, and can be tapered as desired to facilitate insertion into the anatomical soft tissue. The distal end 47b can be tapered in some examples.

The inner central axis 53 can be oriented along the longitudinal direction. The inner dilator body portion 47, and thus the inner dilator member 46, defines an inner external dilation surface 52 that faces tissue and dilates the tissue as the inner dilator member is driven through the tissue toward the target anatomical site. The inner dilator body portion 47, and thus the inner dilator member 46, can further define an internal surface 55 that is opposite the external surface 52. The inner dilator member 46, and in particular the internal surface 55, can define an inner dilator lumen 54 that extends through the inner dilator body portion 47 from the proximal end 47a to the distal end 47b along the inner central axis 53. In one example, the inner central axis 53 can be centrally disposed in the inner dilator lumen 54. Thus, the inner central axis 53 can define a continuous straight line along an entirety of its length.

The inner dilator lumen 54 can be sized approximately equal to the guide member # (see FIG. 3B), such that the inner dilator member can be movable along the guide member # in the proximal direction and guided toward the target anatomical site. Thus, the inner dilator member 46, and in particular the external surface 52 of the inner dilator body portion 47, can expand or dilate, an opening through the tissue that was created by the guide member #, alone or in combination with an instrument that drove the guide wire toward the target anatomical site. In other examples, the inner dilator member 46 can create an opening through the anatomical tissue and establish the path toward the target anatomical site along the desired trajectory. In this regard, the inner dilator body portion 47 can be solid, or the inner dilator member can define the inner dilator lumen 54 as described above.

The inner dilator body portion 47, and thus the inner dilator member 46, can define an inner dilator stop surface 56. In one example, the stop surface 56 can be an external stop surface that is defined by the external surface 52. As will be appreciated from the description below, the stop surface 56 is configured to abut a corresponding stop surface of a second dilator member, which can be defined by the middle dilator member 48 or the outer dilator member 50 if the serial dilation system 30 does not include the middle dilator member 48. In this regard, the inner dilator member 46 can be referred to as a first dilator member, and the associated structure of the inner dilator member 46 can be referred to as a first structure. For instance, the inner central axis 53 can be referred to as a first central axis. The associated structure of the middle or outer dilator members 48 and 50 can be referred to as second structure. As the inner dilator member 46 moves in the proximal direction, the corresponding stop surfaces abut each other, which causes the second dilator to move in the proximal direction along with the inner dilator member 46. Thus, both the inner dilator member 46 and the second dilator can be removed from the access cannula 34 together in a single removal step.

In one example, the external surface 52 of the inner dilator body portion 47 can be inwardly stepped as it extends in the proximal direction so as to define an external shoulder 58. The external shoulder 58 can face the proximal direction, and can define the stop surface 56. Accordingly, the external surface 52 can define a first external cross-sectional dimension IE1 along a first radial direction that is perpendicular to the inner central axis 53 and intersects the central axis 53. The external surface 52 can define a second external cross-sectional dimension IE2 along the first radial direction at a location spaced from the first external cross-sectional dimension in the proximal direction. The second external cross-sectional dimension IE2 can be less than the first external cross-sectional dimension IE1. The external shoulder can be disposed between the first external cross-sectional dimension IE1 and the second external cross-sectional dimension IE2 with respect to the longitudinal direction. In examples where the external surface 52 defines a circle in cross-section, the first and second external cross-sectional dimensions IE1 and IE2 can be configured as diameters. It is recognized, of course, that the external surface 52 can define any suitable size and shape as desired. For instance, the external surface 52 can be symmetrical or asymmetrical about the central axis 53.

Thus, the external surface 52 can define a first portion 52a that defines the first external cross-sectional dimension IE1, and a second portion 52b that defines the second external cross-sectional dimension IE2. The first portion 52a can be spaced from the second portion 52b in the distal direction. The external shoulder 58 can extend radially outward away from the central axis from the second portion 52b to the first portion 52a. Thus, the external shoulder 58 can extend radially outward from the second external cross-sectional dimension IE2 to the first external cross-sectional dimension TEL In one example, the external shoulder 58 can extend in a plane that is perpendicular to the central axis 53, or can extend in any suitable direction having any suitable shape as desired.

In one example, the first external cross-sectional dimension IE1 can extend entirely and continuously about the central axis 53. Thus, stop surface 56 can be configured to abut the stop surface of the second dilator member in any rotational orientation of the inner dilator body portion 47 about the central axis 53. In other examples, the first dilator member 46 can be keyed with the second dilator so as to allow relative translation along the longitudinal direction only when the first dilator member 46 and the second dilator are aligned in a select relative rotational orientation. Thus, the first external cross-sectional dimension IE1 can extend only partially about the central axis in some examples, such as those whereby the stop surface 56 is longitudinally aligned with the corresponding stop surface of the second dilator when the inner dilator member 46 and the second dilator are aligned in the select relative rotational orientation.

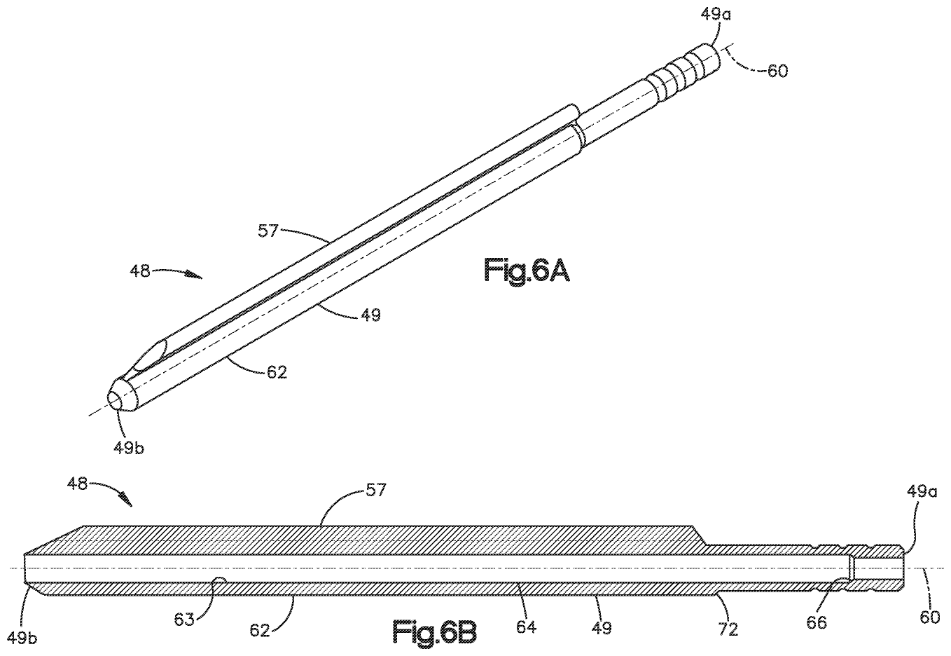

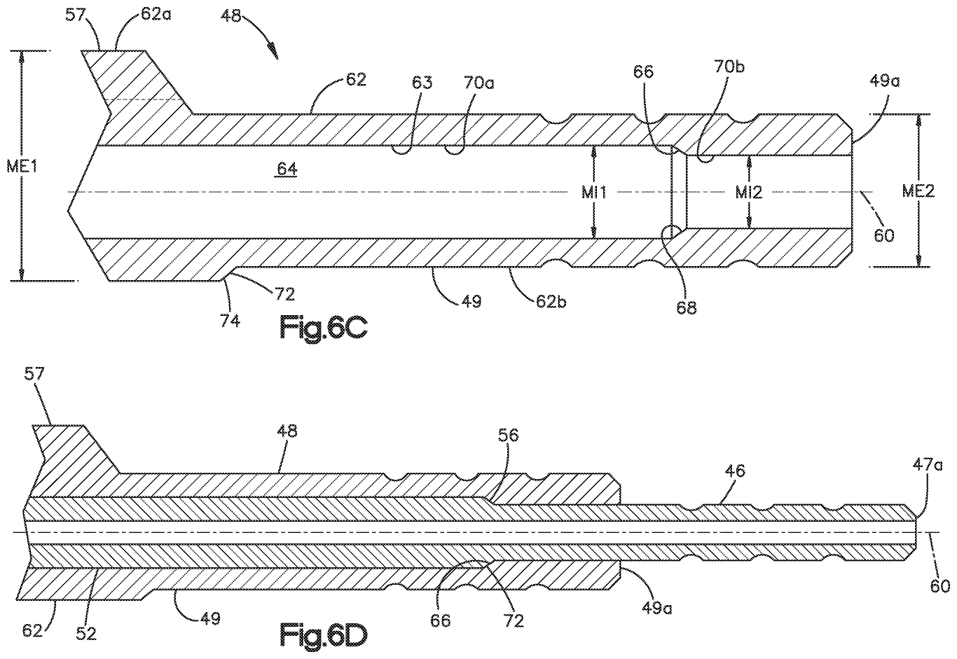

The middle dilator member 48 will now be described with reference to FIGS. 6A-6C. In particular, the middle dilator member 48 includes a middle dilator body portion 49, which can be configured as a middle dilator tube, that extends along a middle central axis 60 that extends from a proximal end 49a of the middle dilator body portion 49 to a distal end 49b of the middle dilator body portion 49. Thus, the proximal direction extends from the distal end 49b to the proximal end 49a. The distal direction extends from the proximal end 49a to the distal end 49b. The distal end 49b can be tapered in some examples. The longitudinal direction can include both the proximal direction and the distal direction. The proximal end 49a can be referred to as a leading end of the middle dilator member 48 with respect to insertion into the soft anatomical tissue. The proximal end 49a can be open so as to fit over the inner dilator member 46. The middle central axis 60 can be oriented along the longitudinal direction. The middle dilator body portion 49, and thus the middle dilator member 48, defines a middle external dilation surface 62 that faces tissue and dilates the tissue as the middle dilator member 48 is driven through the tissue toward the target anatomical site. The middle dilator body portion 49, and thus the middle dilator member 48, can further define an internal surface 63 that is opposite the external surface 62. The external surface 62 is radially outwardly spaced from the internal surface 63. The middle dilator member 48, and in particular internal surface 63 can define a middle dilator lumen 64 that extends through the middle dilator body portion 49 from the proximal end 49a to the distal end 49b along the middle central axis 60. In one example, the middle central axis 60 can be centrally disposed in the middle dilator lumen 64. Thus, the middle central axis 60 can define a continuous straight line along an entirety of its length.

The middle dilator body portion 49, and thus the middle dilator member 48, can define a first middle dilator stop surface 66. The first middle dilator stop surface 66 can be defined by the internal surface 63, and thus can be referred to as an internal stop surface in one example. As will be appreciated from the description below, the first middle dilator stop surface 66 is configured to abut the stop surface 56 of the inner dilator member 46 described above. In one example, the internal surface 63 can be inwardly stepped as it extends in the proximal direction so as to define an internal shoulder 68. The internal shoulder 68 can face the distal direction, and can define the first middle dilator stop surface 66. Accordingly, the internal surface 63 of the middle dilator body portion 49 can define a first internal cross-sectional dimension MI1 along a respective first radial direction that is perpendicular to the middle central axis 60 and intersects the middle central axis 60. The first radial direction of the middle dilator member 48 can be parallel with, coincident with, or angularly offset with respect to, the first radial direction of the inner dilator member 46 described above. The internal surface 63 can define a second internal cross-sectional dimension MI2 along the respective first radial direction at a location spaced from the first internal cross-sectional dimension MI1 in the proximal direction. The second internal cross-sectional dimension MI2 can be less than the first internal cross-sectional dimension MI1. The internal shoulder 68 can be disposed between the first internal cross-sectional dimension MI1 and the second internal cross-sectional dimension MI2 with respect to the longitudinal direction. In examples where the internal surface 63 of the middle dilator body portion 49 defines a circle in cross-section, the first and second internal cross-sectional dimensions MI1 and MI2 can be configured as diameters. It is recognized, of course, that the internal surface 63 can define any suitable size and shape as desired. For instance, the internal surface 63 can be symmetrical or asymmetrical about the middle central axis 60.

Thus, the internal surface 63 can define a first portion 70a that defines the first internal cross-sectional dimension MI1, and a second portion 70b that defines the second internal cross-sectional dimension MI2. The first portion 70a can be spaced from the second portion 70b in the distal direction. The internal shoulder 68 can extend radially outward away from the central axis from the second portion 70b to the first portion 70a. Thus, the internal shoulder 68 can extend radially outward from the second internal cross-sectional dimension MI2 to the first internal cross-sectional dimension MI1. In one example, the internal shoulder 68 can extend in a plane that is perpendicular to the central axis 53, or can extend in any suitable direction having any suitable shape as desired.

Referring now to FIG. 6D, the first internal cross-sectional dimension MI1 of the middle tune 49 can be sized substantially equal to the first external cross-sectional dimension IE1 of the inner dilator body portion 47, such that the inner dilator body portion 47 can nest in the lumen 64 of the middle dilator body portion 49. Thus, the first portion 70a of the internal surface 63 of the middle dilator body portion 49 can receive and ride along the first portion 52a of the external surface 52 of the inner dilator body portion 47. The external surface 52 of the inner dilator body portion 47 can further guide the movement of the middle dilator member 48 along the desired trajectory toward the target anatomical site. Because the external surface 62 of the middle dilator body portion 49 defines an external cross-sectional dimension through the middle central axis 60 that is greater than the first external cross-sectional dimension IE1 of the inner dilator body portion 47, the middle dilator body portion 49 can further dilate the opening through the tissue as the middle dilator member 48 moves through the tissue in the distal direction with respect to the inner dilator member 46. In some examples, the central axes 53 and 60 of the inner dilator body portion 47 and the middle dilator body portion 49 can be substantially coincident or substantially parallel to each other.

When the inner dilator body portion 47 is received in the middle dilator body portion 49, the first stop surface 66 of the middle dilator member 48 is aligned with the stop surface 56 of the inner dilator member 46 along the longitudinal direction. When the middle dilator member 48 is advanced in the distal direction with respect to the inner dilator member 46 to further dilate the anatomical soft tissue, the first stop surface 66 of the middle dilator member 48 is spaced from the stop surface 56 of the inner dilator member 46 in the proximal direction. The middle dilator member 48 can be advanced in the distal direction with respect to the inner dilator member 46 until the respective stop surfaces 66 and 56 abut each other. When the stop surfaces 66 and 56 abut each other, the middle dilator member 48 can no longer translated in the distal direction relative to the inner dilator member 46. Further, the inner and middle dilator members 46 and 48 are coupled with respect to movement in the proximal direction, such that movement of the inner dilator member 46 in the proximal direction causes the middle dilator member 48 to likewise move in the proximal direction. The movement can be a pure translational movement in some examples.

In one example, the first internal cross-sectional dimension MI1 can extend entirely and continuously about the central axis 60. Thus, the first stop surface 66 of the middle dilator member 48 can be configured to abut the stop surface 56 of the inner dilator member 46 in any relative rotational orientation of the second dilator member 48 with respect to the first dilator member 46. In other examples, the first internal cross-sectional dimension MI1 can extend only partially about the central axis 60.

Referring again to FIGS. 6A-6C, the middle dilator body portion 49, and thus the middle dilator member 48, can define a second middle dilator stop surface 72. In one example, the second middle dilator stop surface 72 can be defined by the external surface 62, and thus can be referred to as an external stop surface. As will be appreciated from the description below, the second middle dilator stop surface 72 is configured to abut a corresponding stop surface of a third dilator member, which can be defined by a second middle dilator member if the serial dilation system 30 includes more than one of the middle dilator members 48, or can be defined by the outer dilator member 50 if either 1) the serial dilation system 30 includes a single middle dilator member 48, or the middle dilator member 48 is an outermost one of the middle dilator members 48. The associated structure of the middle or outer dilator members 48 or 50 can be referred to as third structure when the middle or outer dilator member 48 or 50 defines a third dilator member. As the middle dilator member 48 moves in the proximal direction, the corresponding stop surfaces of the middle dilator member 48 and the third dilator abut each other, which causes the third dilator to move in the proximal direction along with the middle dilator member 48. Thus, both the middle dilator member 48 and the third dilator can be removed from the access cannula 34 together in a single removal step. Further, because movement of the inner dilator member 46 in the proximal direction can cause the middle dilator member 48 to move along with the inner dilator member 46 in the proximal direction, movement of the inner dilator member 46 in the proximal direction can cause both the middle dilator member 48 and the third dilator, and all additional dilators of the dilation system 30, if present, to move in the proximal direction.

In one example, the external surface 62 of the middle dilator body portion 49 can be inwardly stepped as it extends in the proximal direction so as to define an external shoulder 74. The external shoulder 74 can face the proximal direction, and can define the second middle dilator stop surface 72. Accordingly, the external surface 62 can define a first external cross-sectional dimension ME1 along a respective second radial direction that is perpendicular to the middle central axis 60 and intersects the central axis 60. In one example, the external shoulder 74 can be spaced in the distal direction with respect to the internal shoulder 68.

The external surface 62 can define a second external cross-sectional dimension ME2 along the second radial direction at a location spaced from the first external cross-sectional dimension ME1 in the proximal direction. The second radial direction can be parallel with, coincident with, or angularly offset with respect to, the first radial dimension. The second external cross-sectional dimension ME2 can be less than the first external cross-sectional dimension ME1. The external shoulder 74 can be disposed between the first external cross-sectional dimension ME1 and the second external cross-sectional dimension ME2 with respect to the longitudinal direction. In examples where the external surface 62 defines a circle in cross-section, the first and second external cross-sectional dimensions ME1 and ME2 can be configured as diameters. It is recognized, of course, that the external surface 62 can define any suitable size and shape as desired. For instance, the external surface 62 can be symmetrical or asymmetrical about the central axis 60.

Thus, the external surface 62 can define a first portion 62a that defines the first external cross-sectional dimension ME1, and a second portion 62b that defines the second external cross-sectional dimension ME2. The first portion 62a can be spaced from the second portion 62b in the distal direction. The external shoulder 74 can extend radially outward away from the central axis 60 from the second portion 62b to the first portion 62a. Thus, the external shoulder 74 can extend radially outward from the second external cross-sectional dimension ME2 to the first external cross-sectional dimension ME1. In one example, the external shoulder 74 can extend in a plane that is perpendicular to the central axis 60, or can extend in any suitable direction having any suitable shape as desired.

In one example, the first external cross-sectional dimension ME1 can extend entirely and continuously about the central axis 60. Thus, second middle dilator stop surface 72 can be configured to abut the stop surface of the third dilator member in any rotational orientation of the middle dilator member 48 about the central axis 60. In other examples, the middle dilator member 48 can be keyed with the third dilator so as to allow relative translation along the longitudinal direction only when the middle dilator member 48 and the third dilator are aligned in a select relative rotational orientation. Thus, the first external cross-sectional dimension ME1 can extend only partially about the central axis 60 in some examples, such as those whereby the second middle dilator stop surface 72 is longitudinally aligned with the corresponding stop surface of the third dilator when the middle dilator member 48 and the third dilator are aligned in the select relative rotational orientation. In one example, the second middle dilator stop surface 72 of the middle dilator member 48 can be spaced in the distal direction with respect to the internal stop surface 66 of the middle dilator member 48. In other examples, the second middle dilator stop surface 72 of the middle dilator member 48 can be spaced in the proximal direction with respect to the internal stop surface 66 of the middle dilator member 48.

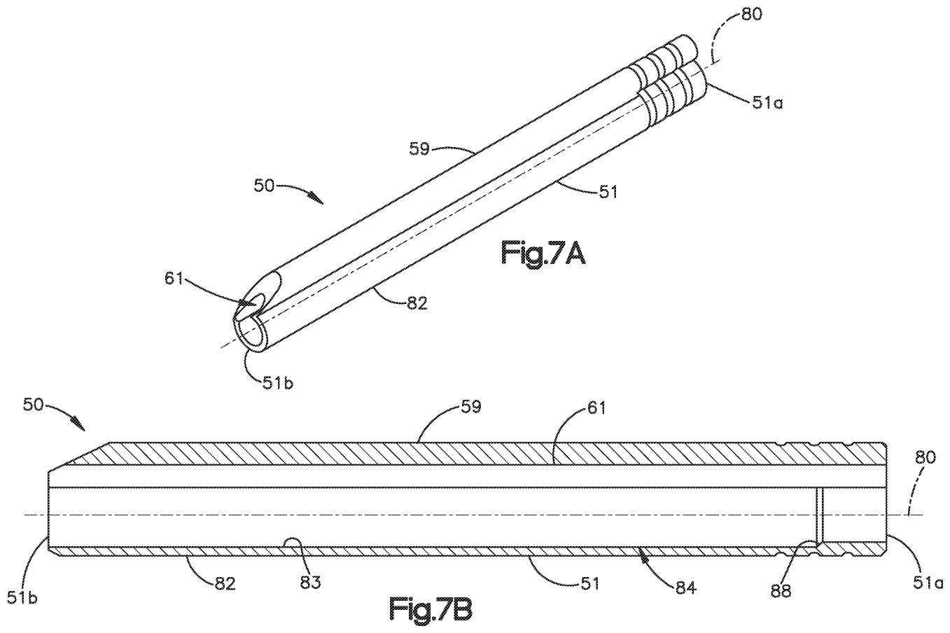

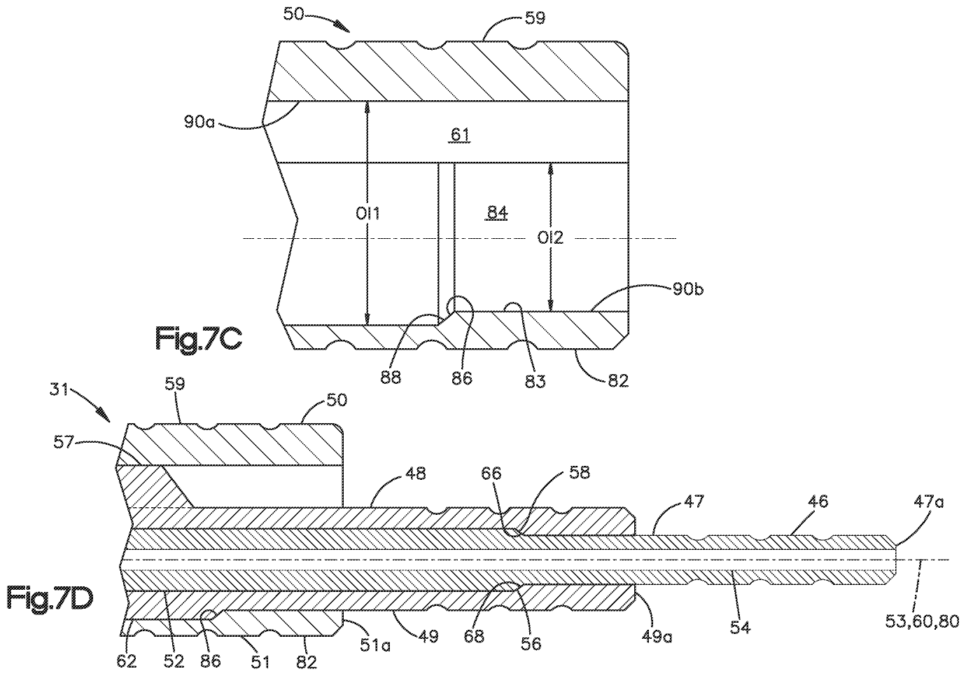

The outer dilator member 50 will now be described with reference to FIGS. 7A-7C. In particular, the outer dilator member 50 includes an outer dilator body portion 51, which can be configured as an outer dilator tube, that extends along an outer central axis 80. The central axis extends from a proximal end 51a of the outer dilator body portion 51 to a distal end 51b of the outer dilator body portion 51. Thus, the proximal direction extends from the distal end 51b to the proximal end 51a. The distal direction extends from the proximal end 51a to the distal end 51b. The longitudinal direction can include both the proximal direction and the distal direction. The distal end 51b can be referred to as a leading end of the outer dilator member 50 with respect to insertion into the soft anatomical tissue. The distal end 51b can be tapered in some examples. The proximal end 51a can be open so as to fit over the middle dilator member 48. The outer central axis 80 can be oriented along the longitudinal direction.

The outer dilator body portion 51, and thus the outer dilator member 50, defines an outer external dilation surface 82 that faces tissue and dilates the tissue as the outer dilator member 50 is driven through the tissue toward the target anatomical site. The outer dilator member 50 defines an external dilation surface 82 that can be sized larger than that an external dilation surface 62 of the middle dilator member 48, which can be sized larger than an external dilation surface 52 of the inner dilator member 46, in a plane that is oriented perpendicular to the longitudinal direction. The outer dilator body portion 51, and thus the outer dilator member 50, can further define an internal surface 83 that is opposite the external surface 82. The external surface 82 is radially outwardly spaced from the internal surface 83. The outer dilator member 50, and in particular the internal surface 63, can define an outer dilator lumen 84 that extends through the outer dilator body portion 51 from the proximal end 51a to the distal end 51b along the outer central axis 80. In one example, the outer central axis 80 can be centrally disposed in the outer dilator lumen 84. Thus, the outer central axis 80 can define a continuous straight line along an entirety of its length.

The outer dilator body portion 51, and thus the outer dilator member 50, can define an outer dilator stop surface 86. The outer dilator stop surface 86 can be disposed at the internal surface 83 in one example. Thus, the outer dilator stop surface 86 can be referred to as an internal stop surface. In one example, the internal stop surface 86 can be defined by the internal surface 83. The stop surface 86 is configured to abut the second middle dilator stop surface 72 of the middle dilator member 48 described above. In one example, the internal surface 83 of the outer dilator body portion 51 can be inwardly stepped as it extends in the proximal direction so as to define an internal shoulder 88. The internal shoulder 88 can face the distal direction, and can define the stop surface 86. Accordingly, the internal surface 83 can define a first internal cross-sectional dimension OI1 along a respective first radial direction that is perpendicular to the outer central axis 80 and intersects the central axis 80. The first radial direction of the outer dilator member 50 can be parallel with, coincident with, or angularly offset with respect to, the first radial direction of the middle dilator member 48 described above. The internal surface 83 can define a second internal cross-sectional dimension OI2 along the respective first radial direction at a location spaced from the first internal cross-sectional dimension OI1 in the proximal direction. The second internal cross-sectional dimension OI2 can be less than the first internal cross-sectional dimension OI1. The internal shoulder 88 can be disposed between the first internal cross-sectional dimension OI1 and the second internal cross-sectional dimension OI2 with respect to the longitudinal direction. In examples where the internal surface 83 defines a circle in cross-section, the first and second internal cross-sectional dimensions OI1 and OI2 can be configured as diameters. It is recognized, of course, that the internal surface 83 can define any suitable size and shape as desired. For instance, the internal surface 83 can be symmetrical or asymmetrical about the central axis 80.

The internal surface 83 can define a first portion 90a that defines the first internal cross-sectional dimension OI1, and a second portion 90b that defines the second internal cross-sectional dimension OI2. The first portion 90a can be spaced from the second portion 90b in the distal direction. The internal shoulder 88 can extend radially outward away from the central axis from the second portion 90b to the first portion 90a. Thus, the internal shoulder 88 can extend radially outward from the second internal cross-sectional dimension OI2 to the first internal cross-sectional dimension OI1. In one example, the internal shoulder 88 can extend in a plane that is perpendicular to the central axis 80, or can extend in any suitable direction having any suitable shape as desired.

Referring now to FIG. 7D, the first internal cross-sectional dimension OD of the outer dilator body portion 51 can be sized substantially equal to the first external cross-sectional dimension ME1 of the middle dilator body portion 49. As a result, the middle dilator body portion 49 can nest in the lumen 84 of the outer dilator member 50. Thus, the first portion 90a of the internal surface 83 of the outer dilator member 50 can receive and ride along the first portion 62a of the external surface 62 of the middle dilator body portion 49. The external surface 52 of the middle dilator body portion 49 can further guide the movement of the outer dilator member 50 along the desired trajectory toward the target anatomical site. Because the external surface 82 of the outer dilator body portion 51 defines an external cross-sectional dimension through the outer central axis 80 that is greater than the first external cross-sectional dimension ME1 of the middle dilator member 48, the outer dilator member 50 can further dilate the opening through the tissue as the outer dilator member 50 moves through the tissue in the distal direction with respect to the middle dilator member 48. In some examples, the central axes 60 and 80 of the middle dilator member 48 and the outer dilator member 50 can be substantially coincident or substantially parallel to each other.

When the middle dilator member 48 is received in the outer dilator member 50, the internal stop surface 86 of the outer dilator body portion 51 is aligned with the second middle dilator stop surface 72 of the middle dilator body portion 49 along the longitudinal direction. When the outer dilator member 50 is advanced in the distal direction with respect to the middle dilator member 48 to further dilate the tissue, the stop surface 86 is spaced from the second middle dilator stop surface 72 in the proximal direction. The outer dilator member 50 can be advanced in the distal direction with respect to the middle dilator member 48 until the respective stop surfaces 86 and 72 abut each other. When the stop surfaces 86 and 72 abut each other, the outed dilator member 50 can no longer translate relative to the middle dilator member 48 in the distal direction. Further, the middle and outer dilator members 48 and 50 are coupled with respect to movement in the proximal direction, such that movement of the middle dilator member 48 in the proximal direction causes the outer dilator member 50 to likewise move in the proximal direction. The movement can be a pure translational movement.

In one example, the internal cross-sectional dimension Oil can extend entirely and continuously about the central axis 80. Thus, stop surface 86 of the outer dilator member 50 can be configured to abut the external stop surface 82 of the middle dilator member 48 in any relative rotational orientation of the outer dilator member 50 with respect to the middle dilator member 48. In other examples, the first internal cross-sectional dimension OI1 can extend only partially about the central axis 60.

With continuing reference to FIGS. 3D, 6A, and 7A, the access cannula 34 can have the radial bump-out 41 that extends from the tubular body portion 36 along the radial direction, which can be oriented perpendicular with respect to the central axes 53, 60, and 80. Thus, one or more of the serial dilators 31 can include a respective radial bump-out so as to similarly dilate the anatomical soft tissue to define a radial-bump out prior to insertion of the access cannula 34. Thus, the dilated radial bump-out in the anatomical soft tissue can be configured to receive the radial bump-out 41 of the access cannula 34. In one example, the middle dilator member 48 can include a middle radial bump-out 57 that extends out from the middle dilator body portion 49 in a direction away from the middle central axis 60. The middle radial bump-out 57 can extend along a majority up to a substantial entirety of the length of the middle dilator body portion 49 along the central axis 60. The outer dilator member 50 can similarly include an outer radial bump-out 59 that extends out from the outer dilator body portion 51 in a direction away from the middle central axis 60. The outer radial bump-out 59 can extend along a majority up to a substantial entirety of the length of the middle dilator body portion 49 along the central axis 60. The outer radial bump-out can further define an internal outer bump-out lumen 61 sized to receive the middle radial bump-out 57 when the outer dilator member 50 is driven over the middle dilator member 48. The middle radial bump-out 57 can be devoid of internal lumens that are open to the anatomical soft tissue along the longitudinal direction in some examples.

During operation, the middle radial bump-out 57 can define and dilate a radial bump-out in the dilated anatomical soft tissue when the middle dilator member 48 is driven distally into the anatomical soft tissue. The outer radial bump-out 59 can expand or dilate the radial bump-out in the dilated anatomical soft tissue when the outer dilator member 50 is driven distally over the middle dilator member 48 into the anatomical soft tissue. Thus, the radial bump-out 41 of the access cannula 34 can be driven through the expanded radial bump-out in the dilated anatomical soft tissue when the access cannula 34 is driven distally over the outer dilator member 50. It should be appreciated that, in some examples, the inner dilator member 46 can also define a respective radial bump-out if desired.

During operation, referring to FIGS. 3-7D generally, the guide member 26 can be driven in the distal direction along the desired trajectory through Kambin's triangle and toward or into the intervertebral space 13. The serial dilators 31 have been preassembled such that the outer dilator member 50 is coupled to the middle dilator member 48, which in turn is coupled to the inner dilator member 46. In particular, the outer dilator member 50 receives the middle dilator member 48, which receives the inner dilator member 46. That is, the inner dilator member 46 is received in the middle dilator lumen 64 of the middle dilator tube 48. The middle dilator member 48 is received in the outer dilator lumen 84 of the outer dilator member 50.

Next the inner dilator member 46 can be driven over the guide member 26 in the distal direction, and thus along the desired trajectory. The inner dilator lumen 54 receives the guide member 26 as the inner dilator member 46 is driven over the guide member 26. In one example, the inner dilator member 46 can be driven to a location whereby the inner dilator member 46 does not extend through Kambin's triangle. The external dilation surface 52 of the inner dilator member 46 further expands or dilates an opening in the anatomical soft tissue that was created by the guide member 26. It is appreciated that the inner dilator member 46 is driven in the distal direction with respect to the middle dilator member 48 and the outer dilator member 50. The inner dilator member 46 can be driven to a desired position and retained in position by an external handle or any suitable alternative fixture as desired.

Next the middle dilator member 48 can be driven over the inner dilator member 46 in the distal direction, and thus along the desired trajectory. The middle dilator lumen 64 receives the external dilation surface 52 of the inner dilator member 46 as the middle dilator member 48 is driven over the inner dilator member 46. In one example, the middle dilator member 48 can be driven to a location whereby the inner dilator member 46 does not extend through Kambin's triangle. It is appreciated that the middle dilator member 48 is driven in the distal direction with respect to the inner dilator member 46 and the outer dilator member 50. The external dilation surface 62 of the middle dilator member 48 is larger than the external dilation surface 52 of the inner dilator member 46, and thus further expands or dilates the opening in the anatomical soft tissue that was expanded by the inner dilator member 46. Additionally, the middle radial bump-out 57 can create a corresponding bump-out opening in the anatomical soft tissue. The middle dilator member 48 can be driven in the distal direction relative to the inner dilator member 46 until the middle dilator internal stop surface 66 abuts the inner dilator stop surface 56 at the external surface 52 of the inner dilator member 46.

Next the outer dilator member 50 can be driven over the middle dilator member 48 in the distal direction, and thus along the desired trajectory. The outer dilator lumen 84 receives the external dilation surface 62 of the middle dilator member 48 as the outer dilator member 50 is driven over the middle dilator member 48. In one example, the outer dilator member 50 can be driven to a location whereby the outer dilator member 50 does not extend through Kambin's triangle. In one example, the inner dilator member 46, the middle dilator member 48, and the outer dilator member 50 can terminate at respective locations adjacent to Kambin's triangle. It is appreciated that the outer dilator member 50 is driven in the distal direction with respect to the inner dilator member 46 and the middle dilator member 48. The external dilation surface 82 of the outer dilator member 50 is greater than the external dilation surface 52 of the inner dilator member 46, and thus further expands or dilates the opening in the anatomical soft tissue that was expanded by the middle dilator member 48. Additionally, the outer radial bump-out 59 can enlarge the bump-out opening created (or in some examples enlarged) by the middle radial bump-out 57.

The outer dilator member 50 can be driven in the distal direction until the outer dilator stop surface 86 abuts the second middle dilator stop surface 72 at the external surface 62 of the middle dilator member 48. At this point, the outer dilator member 50 is unable to be driven distally relative to the middle dilator member 48, which in turn is unable to be driven distally relative to the inner dilator member 46. Because the inner dilator member 46 can be positionally fixed with respect to movement in the distal direction, the serial dilators 31 are unable to be further advanced in the distal direction.

Finally, the access cannula 34 can be driven over the outer dilator member 50 in the distal direction, and the serial dilators 31 can be removed from the internal lumen 39. The guide member 26 can also be removed. To remove the serial dilators 31, a select one of the dilator members 46-50 is moved in the proximal direction, which causes at least one or more up to all of the other dilator members 46-50 to move with the select one of the dilator members in the proximal direction. In one example, the select one of the dilator members 46-50 is defined by the inner dilator member 46. Thus, a proximal force is applied to the inner dilator member 46 that urges the inner dilator member 46 to move in the proximal direction and out of the anatomical soft tissue.

In particular, when a force in the proximal direction is applied to the inner dilator member 46 sufficient to cause the inner dilator member 46 to move in the proximal direction, the stop surface 56 of the inner dilator member 46 abuts the internal stop surface 66 of the middle dilator member 48, thereby coupling the middle dilator member 48 to the inner dilator member 46 with respect to movement in the proximal direction. Thus, movement of the inner dilator member 46 in the proximal direction drives the middle dilator member 48 to move with the inner dilator member 46 in the proximal direction. Similarly, the second middle dilator stop surface 72 of the middle dilator member abuts the internal stop surface 86 of the outer dilator member 50, thereby coupling the outer dilator member 50 to the middle dilator member 48 with respect to movement in the proximal direction. Thus, movement of the middle dilator member 48 in the proximal direction drives the outer dilator member 50 to move with the middle dilator member 48 in the proximal direction. Therefore, movement of the inner dilator member 46 in the proximal direction drives both the middle dilator member 48 and the outer dilator member 50 to move together in the proximal direction out of the anatomical soft tissue, and in particular out of the internal lumen 39 of the access cannula 34. The access cannula 34 can remain in place when the dilator members 46-50 are removed in the proximal direction.

In one example, the inner dilator member 46 and middle dilator member 48 can be prevented from rotating relative to each other as they translate with respect to each other along the longitudinal direction. Alternatively, along the inner dilator member 46 and middle dilator member 48 can be rotatable with respect to each other about the longitudinal direction as they translate with respect to each other along the longitudinal direction. Similarly, the middle dilator member 48 and outer dilator member 50 can be prevented from rotating relative to each other as they translate with respect to each other along the longitudinal direction. Alternatively, along the middle dilator member 48 and outer dilator member 50 can be rotatable with respect to each other about the longitudinal direction as they translate with respect to each other along the longitudinal direction.

As illustrated at FIGS. 5A-7D generally, the stop surfaces 56, 66, 72, and 86 can be configured as respective proximal stop surfaces that are disposed proximate to the respective proximal ends of the respective dilator members. For instance, each of the stop surfaces 56, 66, 72, and 86 can be spaced in the proximal direction from a respective midplane that extends through the respective dilator member in a direction perpendicular to the longitudinal direction. The midplane is equidistantly spaced from the respective proximal end and the respective distal end. In one example, each of the stop surfaces 56, 66, 72, and 86 can be spaced in the proximal direction from a respective second plane that extends through the respective dilator member in a direction perpendicular to the longitudinal direction. The second plane can be equidistantly spaced from the respective midplane to the respective proximal end of the respective dilator member. In this regard, the stop surface 56 of the inner dilator member 46 can be spaced in the proximal direction with respect to the inner external dilation surface 52. Similarly, the second stop surface 72 of the middle dilator member 48 can be spaced in the proximal direction with respect to the middle external dilation surface 62.

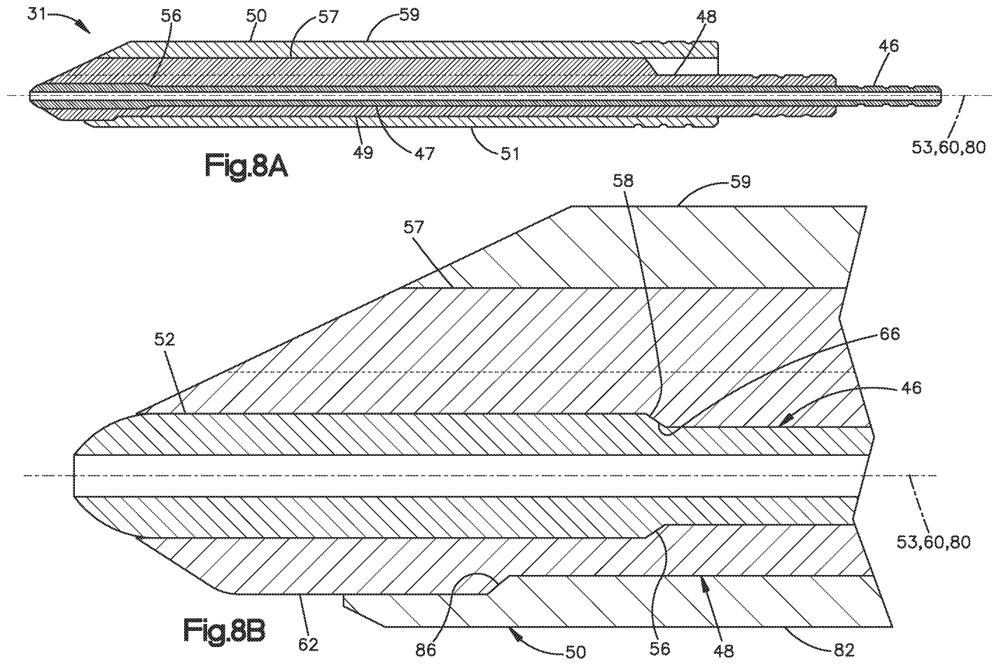

While the serial dilators 31 have been described in accordance with one example, it is recognized that the serial dilators 31 can be constructed in accordance with numerous alternative examples. For instance, referring to FIGS. 8A-8B, the stop surfaces 56, 66, 72, and 86 can be configured as respective distal stop surfaces that are disposed proximate to the respective distal ends of the respective dilator members. For instance, each of the stop surfaces 56, 66, 72, and 86 can be spaced in the distal direction from the respective midplane that extends through the respective dilator member in a direction perpendicular to the longitudinal direction. The midplane is equidistantly spaced from the respective proximal end and the respective distal end. In one example, each of the stop surfaces 56, 66, 72, and 86 can be spaced in the distal direction from the respective second plane that extends through the respective dilator member in a direction perpendicular to the longitudinal direction. The second plane can be equidistantly spaced from the respective midplane to the respective distal end of the respective dilator member.

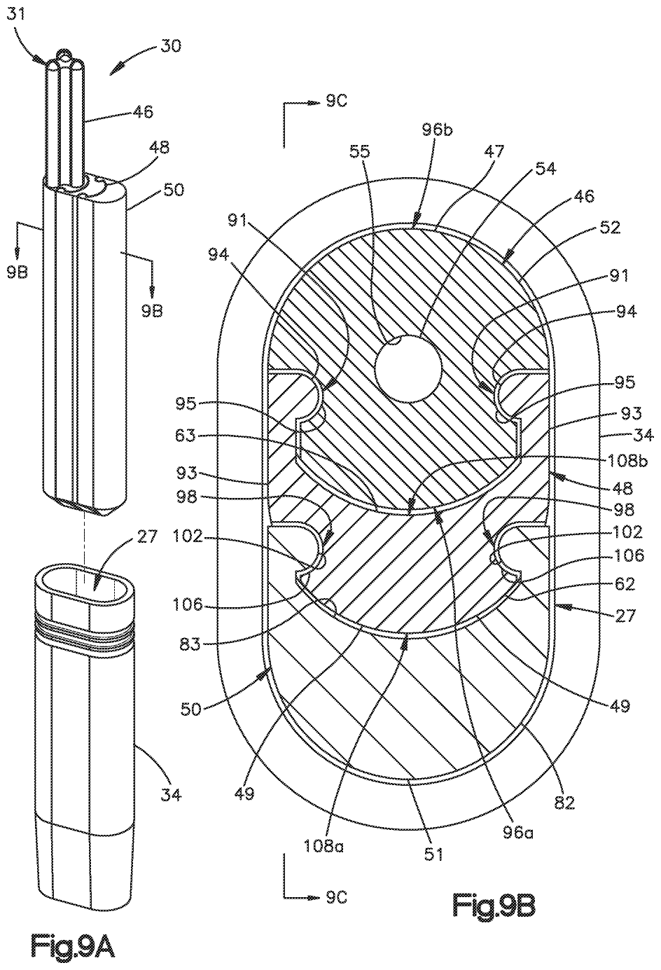

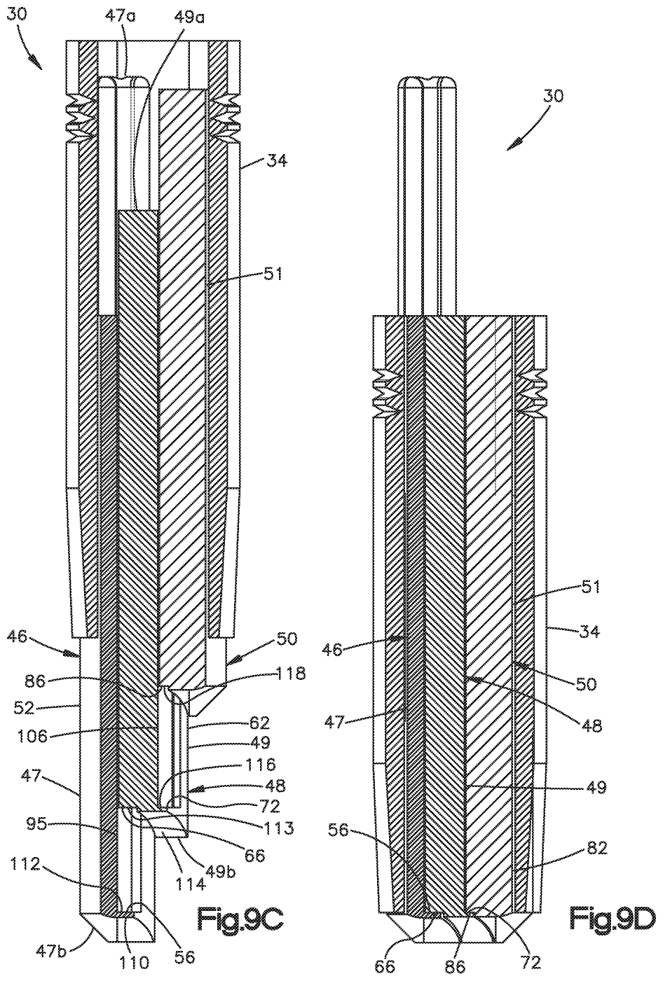

Referring now to FIGS. 9A-9D, the serial dilators 31 can be constructed in accordance with yet another example. For instance, referring to FIGS. 9A-9B in particular, the middle dilator member 48 can translatably mate with the inner dilator member 46 at a respective at least one inner tongue-and-groove interface 91. Thus, the inner dilator member 46 and the middle dilator member 48 can translate with respect to each other along the longitudinal direction. In one example, the middle dilator member 48 can translatably receive the inner dilator member 46 at a respective pair of inner tongue-and-groove interfaces 91. The tongue-and-groove interfaces 91 can define a dovetail in some examples. For example, the middle dilator member 48 can include a middle body portion 49 and at least one middle traveler 94 that is supported by the body portion 49. In particular, the middle dilator member 48 can include at least one middle arm 93 that extends out from the middle body portion 49, and the middle traveler 94 projects out from the middle arm 93 toward the inner dilator member 46. The middle traveler 94 is received in a corresponding inner channel 95 of the inner dilator member 46 to define a respective tongue-and-groove interface 91.

As illustrated, the middle dilator member 48 can include a pair of middle arms 93 that carry respective middle travelers 94 that are received in a pair of corresponding inner channels 95 of the inner dilator member 46 to define a corresponding pair of tongue-and-groove interfaces 91. The middle travelers 94 and inner channels 95 can each be oriented along the longitudinal direction. Further, the middle travelers 94 are translatably received in the inner channels 95. Thus, the middle travelers 94 can travel in the inner channels 95, respectively, as the inner dilator member 46 and the middle dilator member 48 translate relative to each other along the longitudinal direction. The inner channels 95, and thus the middle travelers 94, can be disposed on opposite sides of the central axis 53 of the inner dilator member 46. Thus, the travelers 94 can capture the inner dilator member 46 so as to prevent or limit movement of the middle dilator member 48 with respect to the inner dilator member 46 in a plane that is perpendicular to the longitudinal direction. Further, it should be appreciated that the inner dilator member 46 can be keyed with the middle dilator member 48 so as to allow relative translation along the longitudinal direction only when the inner and middle dilator members 46 and 48 are aligned in a select relative rotational orientation whereby the middle travelers 94 are received in respective ones of the respective inner channels 95. In this regard, the middle dilator member 48 can be prevented from rotating along the central axis with respect to the inner dilator member 46. Thus, it can be said that the inner dilator member 46 and the middle dilator member 48 are rotatably fixed to each other such that the respective stop surfaces are aligned with each other along the longitudinal direction.

The middle dilator member 48 can further receive the inner dilator member 46. For instance, the internal surface 63 of the middle dilator member 48 at the middle body portion 49 can face the external dilation surface 52 of the inner dilator member 46 at a first side 96a of inner dilator member 46 but not at a second side 96b of the inner dilator member 46 that is opposite the first side 96a with respect to the central axis 53 of the inner dilator member 46. A first transverse direction T1 is thus defined perpendicular to the longitudinal direction from the second side 96b to the first side 96a, and a second transverse direction T2 opposite the first transverse direction T1 is defined perpendicular to the longitudinal direction from the first side 96a to the second side 96b. The external dilation surface 52 at the first side 96a of the inner dilator member 46 can be convex, and the internal surface 63 of the middle dilator member 48 at the middle body portion 49 can be concave so as to receive the external dilation surface 52 at the first side of the inner dilator member 46. Further, the external dilation surface 52 at the first side 96a of the inner dilator member 46 can nest in the internal surface 63 of the middle body portion 49. The external dilation surface 52 at the first side 96a of the inner dilator member 46 can face the internal surface 63 of the middle body portion 49 in the first transverse direction T1. Conversely, the internal surface 63 of the middle body portion 49 can face the external dilation surface 52 at the first side 96a of the inner dilator member 46 in the second transverse direction T2.

With continuing reference to FIGS. 9A-9D, the outer dilator member 50 can translatably mate with the middle dilator member 48 at a respective at least one outer tongue-and-groove interface 98. Thus, the middle dilator member 48 and the outer dilator member 50 can translate with respect to each other along the longitudinal direction. In one example, the outer dilator member 50 can translatably receive the middle dilator member 48 at a respective pair of outer tongue-and-groove interfaces 98. For example, the outer dilator member 50 can include the outer dilator body portion 51 at least one outer traveler 102 that is supported by the outer dilator body portion 51. In particular, the outer dilator member 50 can include at least one outer arm 104 that extends out from the outer dilator body portion 51, and the outer traveler 102 projects out from the outer arm 104 toward the middle dilator member 48. The outer traveler 102 is received in a corresponding middle channel 106 of the middle dilator member 48 to define a respective tongue-and-groove interface 98. It should thus be appreciated that the middle dilator member 48 is stacked onto the inner dilator member 46 in the first transverse direction T1, thereby increasing a dimension of the serial dilators 31 in the first transverse direction. The outer dilator member 50 is stacked onto the middle dilator member 48 in the first transverse direction T1, thereby further increasing a dimension of the serial dilators 31 in the first transverse direction. In one example, the dimension of the serial dilators 31 is not increased in the second transverse direction T2 when the middle dilator member 48 is stacked onto the inner dilator member 46, and the outer dilator 50 is stacked onto the middle dilator member 48.

As illustrated, the outer dilator member 50 can include a pair of outer arms 104 that carry respective outer travelers 102 that are received in a pair of corresponding middle channels 106 of the middle dilator member 48 to define a corresponding pair of tongue-and-groove interfaces 98. The tongue-and-groove interfaces 98 can define a dovetail in some examples. The outer travelers 102 can be aligned with respective ones of the middle travelers 94 in the first transverse direction T1. Similarly, the middle channels 106 of the middle dilator member 48 can be aligned with respective ones of the inner channels 95 of the inner dilator member 46 in the first transverse direction T1.

The travelers 102 and the middle channels 106 can each be oriented along the longitudinal direction. Further, the travelers 102 are translatably received in the middle channels 106. Thus, the travelers 102 can travel in the middle channels 106, respectively, as the middle dilator member 48 and the outer dilator member 50 translate relative to each other along the longitudinal direction. The middle channels 106, and thus the travelers 102, can be disposed on opposite sides of the central axis 60 of the middle dilator member 48. The central axis 60 of the middle dilator member 48 can be aligned with the central axis 53 of the inner dilator member 46 in the first transverse direction T1. Similarly, the central axis 80 of the outer dilator member can be aligned with the central axis 60 of the middle dilator member 48 and the central axis 53 of the inner dilator member 46 in the first transverse direction T1. The travelers 102 can capture the middle dilator member 48 so as to prevent or limit movement of the outer dilator member 50 with respect to the middle dilator member 48 in a plane that is perpendicular to the longitudinal direction. Further, it should be appreciated that the middle dilator member 48 can be keyed with the outer dilator member 50 so as to allow relative translation along the longitudinal direction only when the middle and outer dilator members 48 and 50 are aligned in a select relative rotational orientation whereby the outer travelers 102 are received in respective ones of the middle channels 106. In this regard, the outer dilator member 50 can be prevented from rotating along the central axis with respect to the middle dilator member 48. Thus, it can be said that the outer dilator member 50 and the middle dilator member 48 are rotatably fixed to each other such that the respective stop surfaces are aligned with each other along the longitudinal direction.

The outer dilator member 50 can further receive the middle dilator member 48. For instance, the internal surface 83 of the outer dilator member 50 at the outer dilator body portion 51 can face the external dilation surface 62 of the middle body portion 49 of the middle dilator member 48 at a first side 108a of middle dilator member 48 but not at a second side 108b of the middle dilator member 48 that is opposite the first side 108a. The first and second sides 108a and 108b are arranged such that the first transverse direction T1 extends from the first side 108a to the second side 108b, and the second transverse direction T2 extends from the second side 108b to the first side 108a. The external dilation surface 62 at the first side 108a of the middle dilator member 48 can be convex, and the internal surface 83 of the outer dilator member 50 at the outer dilator body portion 51 can be concave so as to receive the external dilation surface 62 of the middle dilator member 48. Further, the external dilation surface 62 at the first side 96a of middle body portion 49 of the middle dilator member 48 can nest in the internal surface 83 of the outer dilator body portion 51. It should be appreciated that the external dilation surface 62 at the first side 108a of the middle body portion 49 of the middle dilator member 48 can face the internal surface 83 of the outer dilator body portion 51 in the first transverse direction T1. Conversely, the internal surface 83 of the outer dilator body portion 51 can face the external dilation surface 62 at the first side 96a of the middle body portion 49 of the middle dilator member 48 in the second transverse direction T2. The middle body portion 49 can be disposed between and aligned with the inner dilator member 46 and the outer dilator body portion 51 with respect to the first and second transverse directions T1 and T2.

As described above, the inner dilator member 46 can be translatable with respect to the middle and outer dilator members 48 and 50 in the distal direction, and the middle dilator member 48 can subsequently be translatable with respect to the outer dilator member 50 in the distal direction. The outer dilator member 50 can then be translatable with respect to each of the inner and middle dilator members 46 and 48. In particular, the inner dilator member 46 can include at least one inner shelf 110 that defines a distal end of the at least one inner channel 95. Thus, the at least one inner channel 95 terminates at a respective inner shelf 110 in the distal direction. In one example, the inner dilator member 46 includes a pair of shelves 110 that define the distal end of the respective pair of inner channels 95. Each inner shelf 110 defines a respective proximal surface 112 that defines the inner dilator stop surface 56. Thus, each inner channel 95 can terminate distally at a respective inner dilator stop surface 56.

Each inner dilator stop surface 56 is configured to abut a corresponding stop surface of the middle dilator member 48 or other second dilator member. In particular, each middle traveler 94 can define a respective distal surface 113 that defines the first dilator stop surface 66 of the middle dilator member 48. The respective distal surface 113 can be aligned with the proximal surface 112 of the inner shelf 110 along the longitudinal direction. Thus, during operation, the inner dilator stop surface 56 is configured to abut the first middle dilator stop surface 66, thereby coupling the middle dilator member 48 to the inner dilator member 46 with respect to movement in the proximal direction. As a result, movement of the inner dilator member 46 in the proximal direction drives the middle dilator member 48 to move with the inner dilator member 46 in the proximal direction.

The middle dilator member 48 is further configured to couple to the outer dilator member 50 with respect to movement in the proximal direction. In particular, each middle dilator member 48 can include at least one middle shelf 114 that defines a distal end of the at least one middle channel 106. Thus, the at least one middle channel 106 terminates at a respective middle shelf 114 in the distal direction. In one example, the middle dilator member 48 includes a pair of middle shelves 114 that define the distal end of the respective pair of middle channels 106. Each middle shelf 114 defines a respective proximal surface 116 that defines the second stop surface 72 of the middle dilator member 48. Each middle channel 106 can terminate distally at a respective second middle dilator stop surface 72. Each second middle dilator stop surface 72 is configured to abut a corresponding stop surface of the outer dilator member 50. In particular, each outer traveler 102 can define a respective distal surface 118 that defines the outer dilator stop surface 86 of the outer dilator member 50. The respective distal surface 118 can be aligned with the proximal surface 116 of the middle shelf 114 along the longitudinal direction. Thus, during operation, the second middle dilator stop surface 72 of the middle dilator member is configured to abut the outer dilator stop surface 86, thereby coupling the middle dilator member 48 to the outer dilator member 50 with respect to movement in the proximal direction. As a result, movement of the middle dilator member 48 in the proximal direction drives the outer dilator member 50 to move with the middle dilator member 48 in the proximal direction.

During operation, the serial dilators 31 can be preassembled as described above, such that the outer dilator member 50 is coupled to the middle dilator member 48, which is coupled to the inner dilator member 46. In particular, the middle travelers 94 of the middle dilator member 48 are disposed in the respective inner channels 95 of the inner dilator member 46. The outer travelers 102 are received in the middle channels 106 of the middle dilator member 48. The inner dilator member 46 is driven into the anatomical soft tissue in the distal direction with respect to each of the middle dilator member 48 and the outer dilator member 50 along the desired trajectory toward the target anatomical site, such as a pedicle, or Kambin's triangle. The middle travelers 94 of the middle dilator member 48 travel proximally in the respective inner channels 95 as the inner channels 95 move in the distal direction. Thus, the inner dilator stop surface 56 and the first middle dilator stop surface 66 separate as the inner dilator member 46 is moved in the distal direction.

The inner dilator member 46 can include one or more sensors, such as a neuromonitoring sensor, to guide the inner dilator member 46 along the desired trajectory. For instance, in one example, the sensor can differentiate soft tissue from bone. Alternatively or additionally, the sensor can emit electrical pulses that can be detected when the sensor is closer to a nerve than a predetermined value. The external dilation surface 52 of the inner dilator member 46 can create an opening in the anatomical soft tissue. Alternatively, a guide member can be first introduced along the desired trajectory, and the inner dilator member 46 can receive the guide member to guide the inner dilator member along the desired trajectory, as described above. The external dilation surface 52 of the inner dilator member 46 can thus further expand or dilates an opening in the anatomical soft tissue that was created by the guide member 26. The inner dilator member 46 can be driven to a desired position and retained in position by an external handle or any suitable alternative fixture as desired.

Once the inner dilator member 46 is in position, the middle dilator member 48 can be driven over the inner dilator member 46 in the distal direction, and thus along the desired trajectory. The middle dilator member 48 also travels in the distal direction with respect to the outer dilator member 50. The outer travelers 102 of the outer dilator member 50 travel proximally in the respective middle channels 106 as the middle channels 106 move in the distal direction. Thus, the second middle dilator stop surface 72 of the middle dilator member 48 moves in the distal direction with respect to the outer dilator stop surface 86, thereby causing separation of the stop surfaces 72 and 86. In one example, the middle dilator member 48 can be driven to a location whereby the middle dilator member 48 extends to, but does not extend through, Kambin's triangle. The external dilation surface 62 of the middle dilator member 48 further expands or dilates the opening in the anatomical soft tissue as the middle dilator member 48 travels distally through the anatomical soft tissue. In particular, the opening in the anatomical soft tissue can be dilated in the first transverse direction T1 as the middle dilator member 48 travels in the distal direction. It should be appreciated that the first middle dilator stop surface 66 travels toward the inner dilator stop surface 56 as the middle dilator member 48 travels in the distal direction. The middle dilator member 48 can travel in the distal direction until the first middle dilator stop surface 66 abuts the inner dilator stop surface 56.

Next, the outer dilator member 50 can be driven over the middle dilator member 48 in the distal direction, and thus along the desired trajectory. The outer dilator member 50 also travels in the distal direction with respect to the inner dilator member 46. The outer travelers 102 of the outer dilator member 50 travel distally in the respective middle channels 106 as the outer dilator member 50 travels in the distal direction. Thus, the outer dilator stop surface 86 moves in the toward the second middle dilator stop surface 72 in the distal direction. In one example, the outer dilator member 50 can be driven to a location whereby the outer dilator member 50 extends to, but does not extend through, Kambin's triangle. The external dilation surface 82 of the outer dilator member 50 further expands or dilates the opening in the anatomical soft tissue as the outer dilator member 50 travels distally through the anatomical soft tissue. In particular, the opening in the anatomical soft tissue can be dilated in the first transverse direction T1 as the outer dilator member 50 travels in the distal direction. The outer dilator member 50 can travel in the distal direction until the outer dilator stop surface 86 abuts the second middle dilator stop surface 72.

Finally, the access cannula 34 can be driven over each of the outer dilator member 50, the middle dilator member 48, and the inner dilator member 46 in the distal direction. The access cannula 34 can directly face each of the outer dilator member 50, the middle dilator member 48, and the inner dilator member 46. The serial dilators 31 can be removed from the internal lumen 39. The guide member 26, if present, can also be removed. To remove the serial dilators 31, a select one of the dilator members 46-50 is moved in the proximal direction, which causes at least one or more up to all of the other dilator members 46-50 to move with the select one of the dilator members in the proximal direction. In one example, the select one of the dilator members 46-50 is defined by the inner dilator member 46. Thus, a proximal force is applied to the inner dilator member 46 that urges the inner dilator member 46 to move in the proximal direction and out of the anatomical soft tissue, which causes the middle and outer dilator members 48 and 50 to similarly move in the proximal direction and out of the anatomical soft tissue. Alternatively, in this embodiment and in all embodiments described herein, the dilator members 46-50 can be coupled to each other such that a proximal force can be applied to the outer dilator member 50 that urges the outer dilator member 50 to move in the proximal direction and out of the anatomical soft tissue, which also causes the middle and inner dilator members 48 and 46 to move in the proximal direction and out of the anatomical soft tissue. For instance, the middle and outer dilator members 48 and 50 can be fixed to each other with any suitable mechanical fastener at their proximal ends, such as a screw or the like. Further, the middle and inner dilator members 48 and 46 can be fixed to each other with any suitable mechanical fastener at their proximal ends, such as a screw or the like.

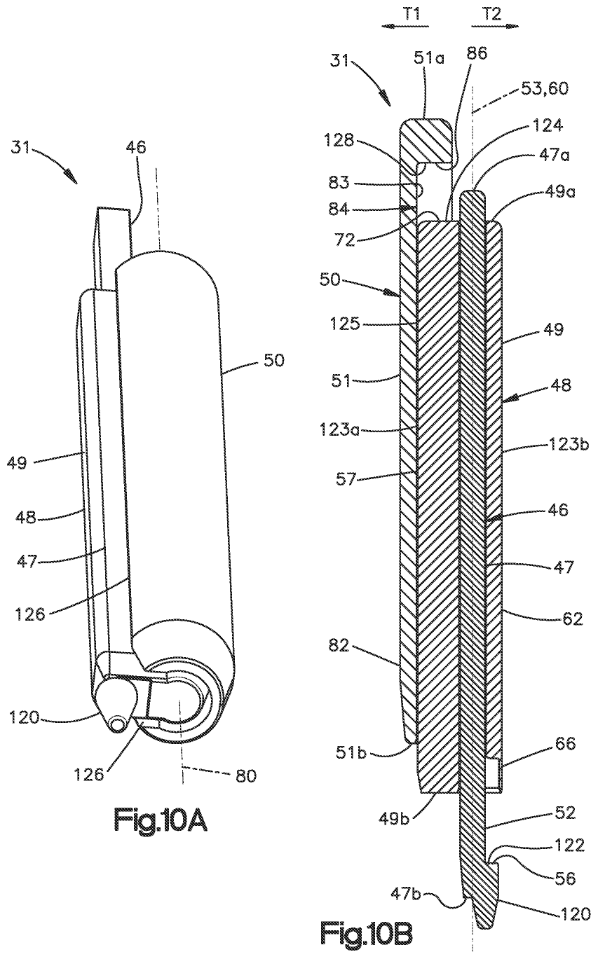

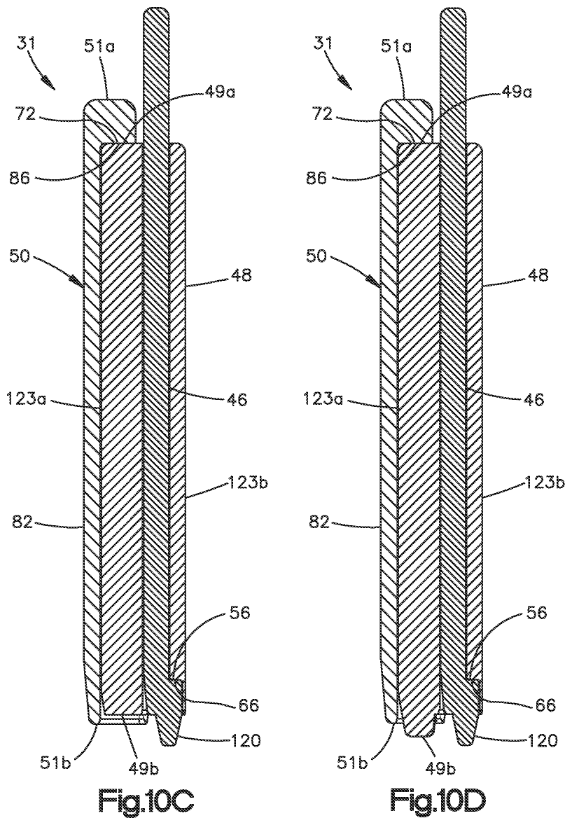

Referring now to FIGS. 10A-10D, it is recognized that the serial dilators 31 can be constructed in accordance with still another embodiment. For instance, in one example the inner dilator member 46 does not define an internal lumen through the inner dilator body portion 47 in this example, and thus is not configured to be driven over a guide member. The inner dilator member 46 can include an inner stop member 120 that projects in the distal direction from the distal end 47b of the inner dilator body portion 47. The stop member 120 can further be jogged with respect to the inner dilator body portion 47 in a direction perpendicular to the middle central axis 60. For instance, the stop member 120 can be jogged in the second transverse direction T2 with respect to the inner dilator body portion 47.

The stop member 120 can include a sensor that guides the inner dilator member 46 through the anatomical soft tissue. For instance, in one example, the sensor can differentiate soft tissue from bone. Alternatively or additionally, the sensor can emit electrical pulses that can be detected when the sensor is closer to a nerve than a predetermined value. The stop member 120 can define a proximal surface 122 that defines the inner dilator stop surface 56 that is configured to abut the complementary first stop surface 66 of the middle dilator member 48 to couple the inner dilator member 46 to the middle dilator member 48 with respect to movement in the proximal direction. Of course, it should be appreciated that the inner dilator member 46 can define an internal lumen that is driven over a guide member in the manner described above.

As described above, the middle dilator member 48 can be translatable with respect to the inner and outer dilator members 46 and 50 in the distal direction. The inner dilator body portion 47 can have a non-circular cross-section in a plane that is perpendicular to the central axis 53. For instance, the cross-section can be rectangular, square, elliptical, or any suitable shape as desired. The middle dilator lumen 64 can receive the inner dilator body portion 47 such that the middle dilator member 48 is translatable with respect to the inner dilator member 46 along the longitudinal direction. Further, the middle dilator lumen 64 can be sized and shaped to correspond to the inner dilator body portion 47. Thus, the middle dilator lumen 64 can be rectangular, square, elliptical, or any suitable shape as desired. In one example, the middle dilator lumen 64 receives the inner dilator body portion 47 such that the middle dilator member 48 is rotatably fixed to each other.

In one example, the distal end 49b of the middle dilator body portion 49 can define the first middle dilator stop surface 66 that faces the distal direction and is aligned with the inner dilator stop surface 56 along the longitudinal direction. In particular, the inner dilator stop surface 56 can be disposed distal of the first middle dilator stop surface 66. Thus, during operation, the inner dilator stop surface 56 is configured to abut the first middle dilator stop surface 66, thereby coupling the middle dilator member 48 to the inner dilator member 46 with respect to movement in the proximal direction. As a result, movement of the inner dilator member 46 in the proximal direction drives the middle dilator member 48 to move with the inner dilator member 46 in the proximal direction in the manner described above. In one example, the first middle dilator stop surface 66 can be recessed in the proximal direction with a remainder of the distal end 49b. Alternatively, the first middle dilator stop surface 66 can be continuous with the remainder of the distal end 49b. Alternatively still, the first middle dilator stop surface 66 can extend distal of the remainder of the distal end 49b.

The middle dilator member 48 is further configured to couple to the outer dilator member 50 with respect to movement in the proximal direction. In particular, the proximal end 49a of the middle dilator body portion 49 defines a proximal surface 124 that defines the second middle dilator stop surface 72. In this regard, the first middle dilator stop surface 66 can be referred to as a distal stop surface disposed at the distal end 49b of the middle dilator member 48, and the second middle dilator stop surface 72 can be referred to as a proximal stop surface disposed at the proximal end of the middle dilator member 48.

The middle dilator member 48 can be asymmetrical with respect to the middle central axis 60. In one example, the middle dilator member 48 can include a first portion 123a that extends further from the middle central axis 60 in the first transverse direction T1 than a second portion 123b that extends from the middle central axis 60 in the second transverse direction T2. In particular, the middle dilator member 48 can include a middle radial bump-out 57 that extends out with respect to the middle dilator body portion 49 in the first transverse direction T1. The middle radial bump-out 57 can define an external middle bump-out surface 125 that is received by the outer dilator lumen 84 such that the outer dilator member 50 is translatable with respect to the middle dilator member 48 along the longitudinal direction.

The outer dilator member 50 can at least partially surround the external middle bump-out surface 125 such that the outer dilator member 50 is rotatably fixed to the middle dilator member 48. In this regard, the outer dilator member 50 can face the middle dilator member 48 at the first 108a of inner dilator member 46 but not the second side 108b of the middle dilator member 48. The middle radial bump-out 57 can define the first side 108a of the inner dilator member 46. The outer dilator member 50 can surround an entirety of the bump-out surface 125 in a plane that is oriented perpendicular to the outer central axis 80, and can define opposed circumferential free ends 126 that each abut opposed sides of the inner dilator portion 47. Thus, the outer dilator lumen 84 can be open along a direction perpendicular to the outer central axis 80. The abutment of the free ends 126 with the opposed sides of the inner dilator portion 47 prevents the outer dilator member 50 from rotating about the middle dilator member 48. The proximal end 51a of the outer dilator body portion 51 can define a distal-facing surface 128 that, in turn, defines the outer dilator stop surface 86. In particular, the distal facing surface 128 can be aligned with the first portion 123a of the middle dilator member 48 along the longitudinal direction. In particular, the second middle dilator stop surface 72 can be disposed distal of the outer dilator stop surface 86. Thus, during operation, the outer dilator stop surface 86 is configured to abut the second middle dilator stop surface 72, thereby coupling the middle dilator member 48 to the outer dilator member 50 with respect to movement in the proximal direction. As a result, movement of the middle dilator member 48 in the proximal direction drives the outer dilator member 50 to move with the inner dilator member 46 in the proximal direction in the manner described above.

In one example, shown at FIG. 10C, the distal end 49b of the middle dilator member 48 at the first portion 123a can be recessed in the proximal direction with respect to the distal end 51b of the outer dilator member 50. Alternatively, referring to FIG. 10D, the distal end 49b of the middle dilator member 48 at the first portion 123a can extend out with respect to the distal end 51b of the outer dilator member 50 in the distal direction. The serial dilators 31 can be operated in the manner described above.

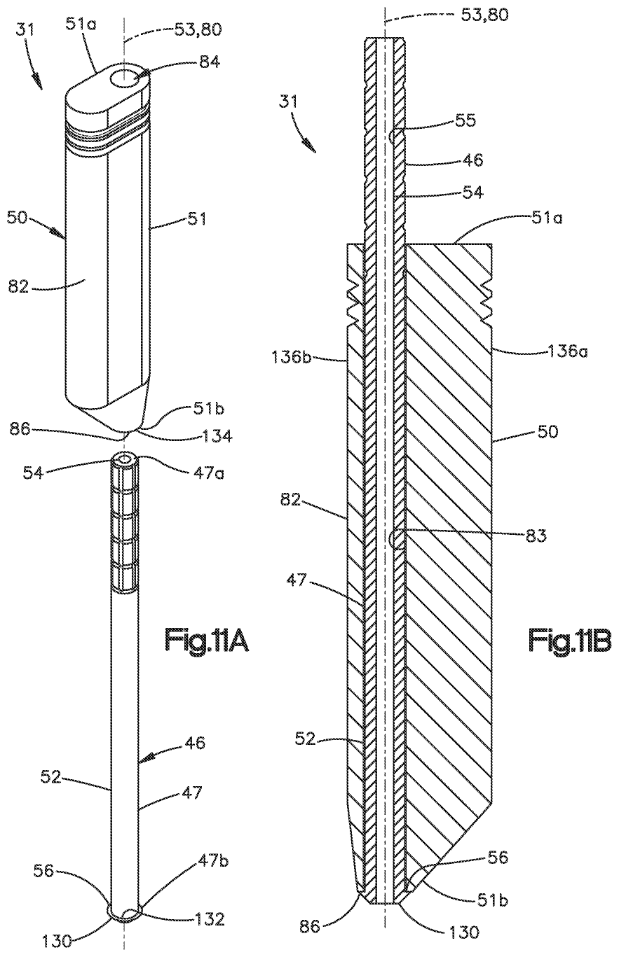

Referring now to FIGS. 11A-11B, the serial dilators 31 can be constructed in accordance with still another example in this example, the serial dilators 31 does not include a middle dilator member. Thus, the serial dilators 31 can include the inner dilator member 46, which defines an innermost one of the dilator members, and the outer dilator member 50 that defines the second dilator member and an outermost one of the dilator members. The inner dilator member 46 includes an inner dilator body portion 47, which can be configured as an inner dilator tube, that extends along the inner central axis 53. The inner dilator member 46 can be symmetrical about the inner central axis 53. The inner dilator member 46 can define the inner dilator lumen 54 that is configured to translatably receive a guide member as described above. The inner dilator member 46 can further include a radial flange 130 that extends out from the external dilation surface 52 away from the inner central axis 53. In one example, the flange 130 can extend out from the distal end 47b of the inner dilator body portion 47. The flange 130 can surround a majority up to an entirety of the external dilation surface 52 in a plane that is oriented perpendicular to the inner central axis 53. The flange 130 can be continuous or discontinuous as it surrounds the majority up to the entirety of the external dilation surface 52. The flange 130 defines a proximal surface 132 that faces the proximal direction. The proximal surface 132 can define the inner dilator stop surface 56. As described above, the inner dilator stop surface 56 is configured to abut a complementary stop surface of the outer dilator member 50 so as to couple the inner dilator member 46 and the outer dilator member 50 with respect to translation in the proximal direction.

The outer dilator member 50 can be asymmetrical about the outer central axis 80. In particular, the outer dilator member 50 can define a first portion 136a that extends further from the outer central axis 80 in a first direction than a second portion 136b that extends from the middle central axis 60 in the second direction that is opposite the first direction. The outer dilator lumen 84 is sized to translatably receive the inner dilator member 46 in the manner described above. Further, the outer dilator member 50 can be rotatable about the inner dilator member about an axis of rotation that can be defined by the inner central axis 53. In one example, the outer dilator member 50 can be rotatable about the inner dilator member 360 degrees about the axis of rotation. During operation, the outer dilator member 50 can be rotated to a position such that the first portion 136a is disposed at a rotational position to create an opening in the soft anatomical tissue that receives the radial bump-out 41 of the access cannula 34 described above.

The distal end 51b of the outer dilator body portion 51 defines a distal surface 134 that faces the distal direction. The distal surface 134 can define the outer dilator stop surface 86. The distal end 51b can taper inwardly toward the central axis 80 to the distal surface 134. The distal end 51b can taper at any suitable angle with respect to the central axis 80 as desired. The proximal surface 132 of the flange 130 is disposed distal of the distal surface 134 of the outer dilator member 50. Thus, the inner dilator member 46 can translate with respect to the outer dilator member 50 in the distal direction. During operation, the inner dilator lumen 54 can receive a guide member to guide the inner dilator member 46 along the desired trajectory in the manner described above. The distal surface 134, and thus the outer dilator stop surface 86, can be aligned with the proximal surface 132 of the flange 130, and thus the inner dilator stop surface 56 along the longitudinal direction as the outer dilator member 50 rotates about the inner dilator member 46. In particular, the stop surfaces 56 and 86 can be aligned with each other at all rotational orientations of the second dilator with respect to the first dilator in a range of 360 degrees of rotation. During operation, as the inner dilator member 46 is moved in the distal direction relative to the outer dilator member 50 into the anatomical soft tissue, the inner dilator stop surface 56 similarly moves away from the outer dilator stop surface 86 in the distal direction. Next, the outer dilator member 50 can be rotated about the inner central axis 53 to a desired rotational position, and subsequently driven in the distal direction relative to the inner dilator member 46 into the soft tissue, thereby further dilating the soft tissue.

The outer dilator member 50 can be driven in the distal direction relative to the inner dilator member 46 until the outer dilator stop surface 86 abuts the inner dilator stop surface 56, which thereby prevents the outer dilator member 50 from being further driven in the distal direction with respect to the inner dilator member 46. Once the access cannula is driven over the outer dilator member 50, a select one of the dilator members 46 and 50 is moved in the proximal direction, which causes the other one of the dilator members 46 and 50 to move with the select one of the dilator members in the proximal direction. In one example, the select one of the dilator members is defined by the inner dilator member 46. Thus, a proximal force is applied to the inner dilator member 46 that urges the inner dilator member 46 to move in the proximal direction and out of the anatomical soft tissue, which also causes the outer dilator member 50 to move in the proximal direction and out of the anatomical soft tissue.

In other examples, the outer dilator member 50 and the inner dilator member 46 can be coupled to each other such that a proximal force can be applied to the outer dilator member 50 that urges the outer dilator member 50 to move in the proximal direction and out of the anatomical soft tissue, which also causes the inner dilator member 46 to move in the proximal direction and out of the anatomical soft tissue. For instance, the inner and outer dilator members 50 can be fixed to each other with any suitable mechanical fastener at their proximal end, such as a screw or the like. Alternatively, the outer dilator member 50 can be stepped so as to define an inner shelf that extends in from the internal surface toward the central axis, and the inner dilator member 46 can be stepped so as to define an outer shelf that extends out from the external surface away from the central axis and engages the inner shelf of the outer dilator member 50, such that a proximal force can be applied to the outer dilator member 50 that urges the outer dilator member 50 to move in the proximal direction and out of the anatomical soft tissue, which also causes the inner dilator member 46 to move in the proximal direction and out of the anatomical soft tissue. The inner and outer shelves can be configured as described and illustrated with respect any shelves of any suitable embodiment.

Although certain embodiments have been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. For instance, the features associated with any of the dilator members described above can be incorporated into any others of the dilator members described above. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A serial dilation system for orthopedic surgery, the serial dilation system comprising:
a first dilator member configured to be driven into anatomical soft tissue, such that a first external surface of the first dilator member dilates an opening in anatomical soft the tissue; and
a second dilator member configured to be advanced along the first external surface in a distal direction and through the anatomical soft tissue, such that a second external surface of the second dilator member further dilates the anatomical soft tissue,
wherein movement of one of the first and second dilator members in a proximal direction opposite the distal direction causes respective first and second stop surfaces of the first and second dilator members to abut each other, thereby causing the other of the first and second dilator members to move in the proximal direction along with the one of the first and second dilator members.

2. The serial dilation system of claim 1, wherein the first dilator member is an inner dilator member that is configured to create the opening in the anatomical soft tissue.

3. The serial dilation system of claim 1, wherein the first dilator member comprises a lumen that is sized to receive a guide member that guides the first dilator to be driven into the anatomical soft tissue along a predetermined trajectory.

4. The serial dilation system of claim 1, wherein the first and second dilator members extend along respective central axes that are oriented along the longitudinal direction, the first and second dilator members define respective external dilation surfaces, and the external dilation surface of the second dilator member is sized larger than the external dilation surface of the first dilator member in a plane that is oriented perpendicular to the longitudinal direction.

5. The serial dilation system of claim 1, wherein the second dilator member is rotatable about the first dilator member, and the stop surfaces of the first and second dilator members are aligned with each other at all rotational orientations of the second dilator with respect to the first dilator in a range of 360 degrees of rotation.

6. The serial dilation system of claim 5, wherein the first dilator member is an innermost one of the dilator members, and the second dilator member is an outermost one of the dilator members.

7. The serial dilation system of claim 5, wherein the first and second dilator members define respective proximal ends and distal ends spaced from the proximal ends in the distal direction, the first dilator member comprises a flange that defines the first stop surface, and the distal end of the second dilator member defines the second stop surface.

8. The serial dilation system of claim 1, further comprising an access cannula configured to be driven over an outermost one of the dilator members prior to movement of the one of the first and second dilator members relative to the other of the first and second dilators in the proximal direction, which causes the other of the first and second dilators to move in the proximal direction along with the one of the first and second dilators.

9. The serial dilation system of claim 1, further comprising a third dilator member that is configured to be advanced along the second external surface in a distal direction and through the anatomical soft tissue so as to cause a third external dilation surface of the third dilator member to further dilate the anatomical soft tissue, wherein movement of the second dilator member in the proximal direction causes respective stop surfaces of the second and third dilator members to abut each other, thereby causing the third dilator member to move in the proximal direction along with the second dilator member.

10. The serial dilation system of claim 9, wherein the first, second, and third dilator members are rotatably fixed to each other.

11. The serial dilation system of claim 9, wherein at least one of the first, second and third dilator members defines a main body portion and a radial bump-out that extends from the main body portion in a direction that is perpendicular to a longitudinal direction, wherein the longitudinal direction includes the proximal direction and the distal direction.

12. The serial dilation system of claim 9, wherein the first stop surface is defined by the external dilation surface of the first dilator member, and the second stop surface is defined by an internal surface of the second dilator member that is opposite the second external dilation surface.

13. The serial dilation system of claim 12, wherein a stop surface of the third dilator member that is defined by an internal surface of the third dilator member that is opposite the third external dilation surface is configured to abut a stop surface of the second dilator member that is defined by the external dilation surface, such that movement of the second dilation member in the proximal direction causes the third dilator member to move in the proximal direction.

14. The serial dilation system of claim 13, wherein the stop surfaces are proximal stop surfaces.

15. The serial dilation system of claim 13, wherein the stop surfaces are distal stop surfaces.

16. The serial dilation system of claim 9, wherein the second dilator member defines an internal surface opposite the second external surface, and the internal surface faces the first external dilation surface of the first dilator member at a first side of the first dilator member but not at a second side of the first dilator member that is opposite the first side.

17. The serial dilation system of claim 16, wherein the third dilator member defines an internal surface opposite the third external surface, and the internal surface of the third dilator member faces the second external dilation surface of the second dilator member at a first side of second dilator member but not at a first side of the second dilator member that is opposite the first side.

18. The serial dilation system of claim 9, wherein the second dilator member defines a pair of travelers that ride in respective channels of the first dilator member, thereby translatably coupling the first and second dilator members to each other, and the third dilator member defines a pair of travelers that ride in respective channels of the second dilator member, thereby translatably coupling the second and third dilator members to each other.

19. The serial dilation system of claim 18, wherein respective shelves terminate the channels of the first and second dilator members and define respective stop surfaces of the first and second dilator members, and the travelers of the second and third dilator members define respective stop surfaces of the second and third dilator members.

20. The serial dilation system of claim 9, wherein the first dilator member defines a stop member that projects in the distal direction and is jogged in a direction that is perpendicular to a longitudinal direction that defines the proximal and distal directions.
